(19) United States
(12) Patent Application Publication
(10) Pub. No.: US 2021/0189374 A1
(43) Pub. Date: Jun. 24, 2021

(54) PRINTABLE MAGNETIC POWDERS AND 3D PRINTED OBJECTS FOR BIONANOCATALYST IMMOBILIZATION

(71) Applicant: ZYMtronix Catalytic Systems, Inc., Ithaca, NY (US)

(72) Inventors: Stephane Cedric Corgie, Ithaca, NY (US); Matthew Stephen Chun, Ithaca, NY (US); Katia Argelia Rodriguez Rivera, Ithaca, NY (US); Maximilian Josef Sanktjohanser, Ithaca, NY (US); Braedon Carter Wong, League City, TX (US)

(73) Assignee: ZYMtronix Catalytic Systems, Inc., Ithaca, NY (US)

(21) Appl. No.: 17/274,164

(22) PCT Filed: Sep. 26, 2019
(86) PCT No.: PCT/US2019/053307

§ 371 Date: Mar. 5, 2021

Related U.S. Application Data
(60) Provisional application No. 62/737,910, filed on Sep. 27, 2018.

(51) Int. Cl. C12N 11/098; C12N 11/14; C12N 9/02; C12N 11/082; C12N 11/096; C12N 9/26; C12N 9/20; C12N 9/08; C12N 9/90; B33Y 70/10; B33Y 80/00

(52) U.S. Cl. CPC C12N 11/098; B22F 10/28; C12N 9/0042; C12N 9/0071; C12Y 106/02004; C12N 11/082; C12N 11/096; C12N 9/2431; C12Y 302/01026; C12N 9/20; C12Y 301/01003; C12N 9/0065; C12Y 111/01007; C12N 9/90; B33Y 70/10; B33Y 80/00; C12N 11/14

(57) ABSTRACT

The invention provides materials, and in particular, magnetic materials, for the universal immobilization of enzymes and enzyme systems. Described herein are highly magnetic and highly porous composite blends of thermoplastics with magnetic particles to form powders, single-layered, or multiple-layered materials that are used as scaffolds for magnetically immobilized enzymes known as bionanocatalysts (BNCs). Designed objects are produced using 3D printing by sintering composite magnetic powders. In some embodiments, Selective Laser Sintering (SLS) is used. The invention provides the use of the material compositions for 3D printing of enzyme supports and flow cells allowing continuous production of, e.g., small molecules.

### CROSS REFERENCE TO RELATED APPLICATIONS

This application claims the benefit of U.S. Provisional Application No. 62/737,910, filed Sep. 27, 2018, which is incorporated herein by reference in its entirety.

### FIELD OF THE INVENTION

The invention provides materials, and in particular, magnetic materials, for the universal immobilization of enzymes and enzyme systems. Described herein are high magnetism and high porosity composite blends of thermoplastics with magnetic particles to form powders, single-layered, or multiple-layered materials that are used as scaffolds for magnetically immobilized enzymes known as bionanocatalysts (BNCs). Designed objects are produced using 3D printing by sintering composite magnetic powders. In some embodiments, Selective Laser Sintering (SLS) is used. The invention provides the use of the composition materials for 3D printing of enzyme supports and flow cells allowing continuous production of small molecules.

### BACKGROUND OF THE INVENTION

Biocatalysis, as a green technology, has become increasingly popular in chemical manufacturing over traditional expensive and inefficient processes. Its applications include the production of food ingredients, flavors, fragrances, commodity and fine chemicals, and active pharmaceuticals. When producing chemicals at industrial scale, however, enzymes can suffer drastic losses in activity and loading causing a significant drop in performance.

Magnetic enzyme immobilization involves the entrapment of enzymes in mesoporous magnetic clusters that self-assemble around the enzymes (level 1). The immobilization efficiency depends on a number of factors that include the initial concentrations of enzymes and nanoparticles, the nature of the enzyme surface, the electrostatic potential of the enzyme, the nature of the nanoparticle surface, and the time of contact. Enzymes used for industrial purposes in biocatalytic processes should be highly efficient, stable before and during the process, reusable over several biocatalytic cycles, and economical.

Mesoporous aggregates of magnetic nanoparticles may be incorporated into continuous or particulate macroporous scaffolds (level 2). The scaffolds may or may not be magnetic. Such scaffolds are discussed in WO2014/055853, WO2017/180383, and Corgie et al., Chem. Today 34(5):15-20 (2016), incorporated by reference herein in their entirety. Highly magnetic scaffolds are designed to immobilize, stabilize, and optimize any enzyme. This includes full enzyme systems, at high loading and full activity, and for the production of, e.g., small molecules.

Selective laser sintering (SLS) is an additive manufacturing (AM) technique that uses a laser as the power source to sinter powdered materials such as nylon or polyamide. A laser automatically aimed at points in space, defined by a 3D model, binds the material together to create a solid structure. It is similar to direct metal laser sintering (DMLS) but differs in technical details. DMLS uses a comparable concept, but in DMLS the material is fully melted rather than sintered. This allows one to manufacture materials with different properties (e.g. crystal structure and porosity). SLS is a relatively new technology that may be expanded into commercial-scale manufacturing processes.

SLS is an additive manufacturing layer technology. It involves high-powered lasers (e.g. a carbon dioxide laser) to fuse small particles of plastic, metal, ceramic, or glass powders into a mass that has a desired three-dimensional shape. The laser selectively fuses powdered material by scanning cross-sections generated from a 3-D digital description of the part (for example from a CAD file or scan data) on the surface of a powder bed. After each cross-section is scanned, the powder bed is lowered by a one-layer thickness, a new layer of material is applied on top, and the process is repeated until the part is completed.

3D printing involves processes in which material is joined or solidified under computer control to create three-dimensional objects. Material is added together (e.g. liquid molecules or powder grains) that is fused together. 3D printing is used in both rapid prototyping and additive manufacturing. Objects can be of almost any shape or geometry and typically are produced using digital model data from a 3D model or another electronic data source such as an Additive Manufacturing File (AMF). Typically, materials are added in sequential layers. There are many different technologies, like stereolithography (SLA) or fused deposit modeling (FDM).

### SUMMARY OF THE INVENTION

The invention provides materials, and in particular, magnetic materials, for the universal immobilization of enzymes and enzyme systems. Described herein are high magnetism and high porosity composite blends of thermoplastics with magnetic particles to form powders, single-layered, or multiple-layered materials that are used as scaffolds for magnetically immobilized enzymes known as bionanocatalysts (BNCs). Designed objects are produced by 3D printing by sintering composite magnetic powders. In some embodiments, Selective Laser Sintering (SLS) is used. The invention provides the use of the composition materials for 3D printing of enzyme supports and flow cells allowing continuous production of small molecules. With the ability to immobilize any enzymes for any processes, the materials disclosed herein, when functionalized with enzymes or enzyme systems, may be used for producing pharmaceutical actives, agricultural actives, flavors and fragrances, fine and commodity chemicals, and food ingredients.

The invention provides a magnetic macroporous powder, comprising a thermoplastic polymer and magnetic microparticles, wherein the powder is operative for immobilizing self-assembled mesoporous aggregates of magnetic nanoparticles.

In some embodiments, the magnetic particles have a size of between about 50-100 μm, 10-50 μm, 5-10 μm, 10 μm, or 5 μm. In other embodiments, the magnetic particles have a size of less than 5 μm, greater than 100 μm or have an average size of about 150 μm, about 75 μm, or about 15 μm.

In some embodiments, the magnetic particles have a concentration of between 0 and 10% by weight. In other embodiments, the magnetic particles have a concentration of 10 to 50% or 50 to 90% by weight.

In some embodiments, the thermoplastic polymer comprises a polymer selected from the group consisting of Polyvinyl alcohol (PVA), Acrylic (PMMA), Acrylonitrile butadiene styrene (ABS), Polyamide including Nylon 6, Nylon 11 and Nylon 12, Polylactic acid (PLA), Polybenzimidazole (PBI), Polycarbonate (PC), Polyether sulfone (PES), Polyoxymethylene (POM), Polyetherether ketone (PEEK), Polyetherimide (PEI), Polyethylene (PE), Polyphenylene oxide (PEO), Polyphenylene sulfide (PPS), Polypropylene (PP), Polystyrene (PS), Polyvinyl chloride (PVC), polytetrafluoroethylene (PTFE), co-polyesters, and chemically functionalized derivatives thereof.

In some embodiments, the magnetic microparticles comprise a magnetic material selected from the group consisting of magnetite (Fe₃O₄), hematite (α-Fe₂O₃), maghemite (γ-Fe₂O₃), a spinel ferrite, lodestone, cobalt, nickel, rare earth, and magnetic composites. In preferred embodiments, the rare earth is neodymium, gadolinium, sysprosium, samarium-cobalt, or neodymium-iron-boron. In other embodiments, the magnetic composite comprises a ceramic, ferrite, or alnico magnet.

The invention provides that, in the magnetic macroporous powders disclosed herein, the thermoplastic polymer and the magnetic microparticles are chemically, thermally, or physically blended.

In some embodiments, the macropores have a size of between 0.5-200 μm.

In some embodiments, the macroporous powders of the invention further comprise cellulose fibers, cellulose nanofibers, glass fibers, or carbon fibers or other reinforcement macro structures.

Some embodiments of the magnetic macroporous powder of the invention further comprise self-assembled mesoporous aggregates of magnetic nanoparticles and an enzyme magnetically immobilized within the mesopores or on their surface.

The invention provides shaped magnetic macroporous scaffolds, comprising the magnetic macroporous powders disclosed herein, wherein a powder has been formed into the shape by three-dimensional (3D) printing. In some embodiments, the shape is a cylinder, an orb, a bead, a strip, a capsule, a cube, a squared rod, a pyramid, a diamond, a lattice, or an irregular shape.

In some embodiments, the shaped magnetic macroporous scaffolds described herein further comprise self-assembled mesoporous aggregates of magnetic nanoparticles. In preferred embodiments, the self-assembled mesoporous aggregates of magnetic nanoparticles further comprise one or more enzymes magnetically immobilized within said mesopores or on the surface of said magnetic nanoparticles.

In some embodiments of the invention, the enzyme is selected from the group consisting of hydrolases, hydroxylases, hydrogen peroxide producing enzymes (HPP), nitralases, hydratases, dehydrogenases, transaminases, ketoreductases (KREDS) ene reductases (EREDS), imine reductases (IREDS), catalases, dismutases, oxidases, dioxygenases, lipoxidases, oxidoreductases, peroxidases, laccases, synthetases, transferases, oxynitrilases, isomerases, gludosidases, kinases, lyases, sucrases, invertases, epimerases, and lipases. In some embodiments of the invention, two or more enzymes are combined.

In other embodiments of the invention, the self-assembled mesoporous aggregates of magnetic nanoparticles comprise microsomes, wherein a first enzyme requiring a diffusible cofactor having a first enzymatic activity is contained within the microsomes, wherein a second enzyme comprising a cofactor regeneration activity is magnetically-entrapped within the mesopores, wherein the cofactor is utilized in the first enzymatic activity; wherein the first and second enzymes function by converting a diffusible substrate into a diffusible product; and wherein the magnetic nanoparticles are magnetically associated with the magnetic macroporous scaffold.

In other embodiments of the invention, the self-assembled mesoporous aggregates of magnetic nanoparticles comprises a first enzyme requiring a diffusible cofactor having a first enzymatic activity; a second enzyme comprising a cofactor regeneration activity; wherein the cofactor is utilized in the first enzymatic activity; wherein the first and second enzymes are magnetically-entrapped within the mesopores formed by the aggregates of magnetic nanoparticles and the first and second enzymes function by converting a diffusible substrate into a diffusible product. In preferred embodiments, the first enzyme is an oxidative enzyme. In more preferred embodiments, the oxidative enzyme is a Flavin-containing oxygenase and further comprising a third enzyme having a co-factor reductase activity that is co-located with the first enzyme.

In a more preferred embodiment, the oxidative enzyme is a P450 monooxygenase and the composition further comprises a third enzyme having a co-factor reductase activity that is co-located with the first enzyme. In a more preferred embodiment, the P450 monooxygenase and the third enzyme are comprised within a single protein. In a more preferred embodiment, the single protein comprises a bifunctional cytochrome P450/NADPH—P450 reductase. In a more preferred embodiment, the single protein has BM3 activity and has at least a 90% sequence identity to SEQ ID NO:1.

In some embodiments of the invention, the macroporous scaffolds disclosed herein are formed in a shape suited for a particular biocatalytic process.

The invention provides a method of making a shaped magnetic macroporous scaffold comprising the magnetic macroporous powders disclosed herein, comprising additively manufacturing (AM) the shaped magnetic macroporous scaffold using a three-dimensional (3D) printer, wherein the shape is taken from a 3D model. In some embodiments, the 3D model is an electronic file. In preferred embodiments, the electronic file is a computer-aided design (CAD) or a stereolithography (STL) file.

In some embodiments, the AM is Fused Filament Fabrication (FFF) or Selective Laser Sintering (SLS).

In some embodiments of the invention, the macropores are formed using a soluble agent selected from the group consisting of a salt, a sugar, or a small soluble polymer. The soluble agent is removed with a solvent.

The invention provides a method of making a device for catalyzing an enzymatic reaction, comprising combining a shaped magnetic macroporous scaffold with self-assembled mesoporous aggregates of magnetic nanoparticles and an enzyme, wherein the enzyme is magnetically immobilized within the mesopores.

The invention provides a method of catalyzing a reaction between a plurality of substrates, comprising exposing the magnetic macroporous powders disclosed herein comprising an enzyme to the substrates under conditions in which the enzyme catalyzes the reaction between the substrates.

The invention provides a method of catalyzing a reaction between a plurality of substrates, comprising exposing the shaped magnetic macroporous scaffold disclosed herein comprising an enzyme to the substrates under conditions in which the enzyme catalyzes the reaction between the substrates.

In other embodiments, the reaction is used in the manufacture of a pharmaceutical product, a medicament, a food product, a flavor, a fragrance, a sweetener, an agrochemical, an antimicrobial agent, a toxin, a detergent, a fuel product, a biochemical product, a paper product, or a plastic product. In other embodiments, the reaction is used in a process for removing a contaminant from a solution. In preferred embodiments, the solution is an aqueous solution, a solvent, or an oil.

In some embodiments, the methods of manufacturing disclosed herein further comprise the step of removing the mesoporous aggregates and replacing them with a fresh preparation of mesoporous aggregates. In other embodiments, the method is carried out using flow cell and continuous manufacturing.

### BRIEF DESCRIPTION OF THE DRAWINGS

SEM images of a polypropylene magnetite composite extruded composite after: (FIG. 1A) cryogenic size reduction and sieving through a 100 micron opening; (FIG. 1B) selective laser sintering at a chamber temperature of 105° C., surface temperature of 125° C., and a laser speed of 650 mm/s; and (FIG. 1C) enzyme immobilization of the sintered scaffold (cross) showing the BNCs (invertase enzyme) magnetically captured on the surface.

SEM images of a Nylon 12 magnetite composite produced through a dissolution coating process. FIG. 2A shows a backscatter detector. FIG. 2B shows it at 10,000 times magnification.

FIG. 3 shows an article size analysis of a polypropylene magnetite extruded composite after cryogenic size reduction.

Sintering of 3D printing objects for enzyme immobilization. 3D renderings are shown for a strip (FIG. 4A), a bead (FIG. 4B), a cross (FIG. 4C), and a static mixing element (FIG. 4D). The corresponding SLS printed objects are shown in FIGS. 4E, 4F, 4G, and 4H. They were printed using a 110° C. chamber temperature, 125° C. fabrication surface temperature, and a 650 mm/s laser speed. FIG. 4I depicts a 3D render of a printed bar for enzyme immobilization. FIG. 4J depicts the SLS printed bar.

Immobilization of invertase. FIG. 5A shows the immobilization yields of invertase on 3D printed polypropylene-magnetite scaffolds (Polypropylene-magnetite cross and bead). Immobilization conditions: 500 μg/mL NP pH 3. FIG. 5B shows the relative activity of free and immobilized invertase (100 μg/mL) for the hydrolysis of raffinose after 5 minutes, as determined by the reducing-sugar PAHBAH assay.

Immobilization of lipase. FIG. 6A shows the immobilization yields of CALB on 3D printed scaffolds (Polypropylene-magnetite cross and bead). Immobilization conditions: 250 μg/mL NP pH 3. FIG. 6B shows the relative activity of free and immobilized CALB (100 μg/mL) for the hydrolysis of p-NPL after 4 minutes as determined by colorimetric assay.

Re-use of the 3D printed scaffolds on a polypropylene-magnetite cross and beads. FIG. 7A shows the relative activity of free CALB (100 μg/mL) and previously immobilized crosses and beads (post-wash in boiling pH 2 water for 10 mins) for the hydrolysis of p-NPL after 4 minutes as determined by colorimetric assay. FIG. 7B shows the re-immobilization yields of CALB on 3D printed scaffolds (cross and bead). Immobilization conditions were 250 μg/mL NP at pH 3. FIG. 7C shows the relative activity of free CALB and re-immobilized CALB on washed scaffolds as determined by p-NPL hydrolysis.

FIG. 8 shows a plot of a flow-rate, F, (mL/min) from a sucrose reaction mixture in a flow cell of immobilized invertase (modeled as a PBR) as a function of various initial sucrose concentrations, So. It also shows the conversion of sucrose into glucose and fructose, X.

FIG. 9 shows the relative activity of immobilized HRP to free enzyme normalized for immobilization yield for the co-oxidation of 4-AAP and phenol as determined by colorimetric assay.

FIG. 10: A 3D printed flow cell set up with a syringe pump. HRP was immobilized onto the flow cell and the activity was confirmed by the colorimetric assay for co-oxidation of 4-AAP and phenol.

### DETAILED DESCRIPTION OF THE INVENTION

The present invention provides compositions and methods for supporting and enhancing the effectiveness of BNC's. This is accomplished, for the first time, using powders, single-layered, and multi-layered forms that are highly magnetic, highly-porous thermoplastic blends. They may be formed using 3D printing that involves sintering composite magnetic powders.

The invention has many benefits over the prior art. It enables the universal immobilization of enzymes for small molecule syntheses. The materials may contain large macropores or a high magnetic surface area for BNC immobilization. Flexible compositions for sintered magnetic scaffolds can be made with any meltable thermoplastics and magnetic material composition. The materials can be made resistant to solvents.

The process is flexible and tunable to manufacture objects using 3D designs that magnetically capture the BNCs. It is scalable from micro-structures to flow cells for biocatalytic reactors. A large surface area may result from the sintering process itself. Materials can also be recycled by removing the BNCs and then re-functionalized them for repeated use.

In some embodiments, thermoplastics such as polypropylene, polyethylene, nylon, and polylactic acid (PLA) are blended with magnetic materials (e.g. magnetite MMP) via melting/extrusion or via coating of the magnetic material by dissolving the plastic in a solvent. In other embodiments, the powders are sintered by a laser using SLS. Porosity may be formed during SLS.

In other embodiments, an extruded composite material is size reduced via cryomilling or another form of milling. In other embodiments, composite powders are sieved to an ideal particle size. In preferred embodiments, the particle sizes are 60+/−20 μm.

Powders or 3D printed objects can be functionalized with BNCs containing one or more enzymes or enzyme systems. BNCs are magnetically trapped at the surface of the powders or 3D printed objects.

Composite powders may also be optimized for flowability. In some embodiments, 3D objects can be printed to optimize flow within to be used in flow reactors.

In some embodiments, 3D objects and composite powders can be washed from the BNCs by an acid wash, rinsed with water, and then re-functionalized with fresh BNCs.

Highly magnetic scaffolds (Macroporous Magnetic Scaffolds or MMP) are designed to immobilize, stabilize and optimize any BNCs containing enzymes. This includes full enzyme systems at high loading and full activity for the production of small molecules. By combining natural or engineered enzymes with cofactor recycling systems, the scaffolds allow one to scale up biocatalysis to innovations to manufacturing scale and production.

MMP made of thermoplastic and magnetic materials of the invention can take the form of magnetic powders that are suitable for use in sequential batch reactions. For flow chemistry application, these powders can be 3D printed by SLS as structures, as functional objects, or as flow cells or plate reactors. High surface areas allow one to maximize the enzyme loading and flow can be engineered within the materials to enable biocatalysis at maximal productivity.

SLS can be used to process nearly any kind of material from metals, ceramics, plastics, and combinations thereof, for tailor-made composite materials. It is critical, however, that the material is available in fine powder form and that the powder particles are operative to fuse when exposing them to heat (Kruth et al., Assembly Automation 23(4):357-371(2003), incorporated by reference herein in its entirety).

When the material lacks those features, or is prone to phase transitions at the temperature range or conditions of the sintering process, the addition of a sacrificial binder can make this process still feasible for that material. Commonly, polymers are used as sacrificial binders in order to expand the range of materials suitable for this technology. After sintering, the sacrificial binder can be either removed by thermal decomposition or kept as part of the composition.

This concept applies to magnetite that loses its permanent magnetic properties above 585° C. This is significantly lower than its melting temperature (1538° C.). Another advantage of using a polymeric matrix to incorporate magnetite particles is that the former can act as a protective barrier to prevent oxidation and corrosion as well as aiding to disperse the magnetite particles. Also, magnetite can mechanically reinforce the polymer. (Shishkovsky et al., Microelectronic Engineering 146:85-91 (2015), incorporated by reference herein in its entirety).

Laser sintering of plastic parts is one of two additive manufacturing processes used for Rapid Manufacturing (Wegner, Physics Procedia 83:1003-1012 (2016), incorporated by reference herein in its entirety). There are several polymer properties that determine its capability to be sintered and produce good quality 3D objects. These include structural properties such crystalline structure (i.e. thermal properties such as Tm, Tg, and Tc), mechanical properties (Young's modulus and elongation at break, etc.), density, particle size, and shape.

In SLS, the temperature-processing window is determined from the difference between the melting and crystallization temperatures of the polymer. For instance, nylon 12 (PA 12) has one of the highest operational windows and is thus a widely used SLS material. In theory, the higher this value is, the easier the material can be sintered. In practice, however there are many more parameters that can still make this process difficult for any specific polymer (Shishkovsky et al., Microelectronic Engineering 146:85-91 (2015), incorporated by reference herein in its entirety). In order to prevent curling of the sintered part, a low polymer crystallization rate is desired together with a melt index that provides a suitable rheology and surface tension.

Additionally, the bulk density, particle shape, and size distribution of the powder are key factors (Wegner, Physics Procedia 83:1003-1012 (2016), incorporated by reference herein in its entirety). It has been determined that the optimal particle size range is about 40 to about 90 microns. Smaller particles prevent flowability and their rapid vaporization is detrimental to the optical sensors of the sintering device. This can fog the device and lead to inaccurately sintered parts (Goodridge et al. Materials Science 57:229-267 (2012), incorporated by reference herein in its entirety). The powders should have good flowing properties and preferably an approximately round particle shape. This allows good powder spreading during the process. High heat conductivity of the material is desired at the CO₂ laser beam wavelength (10.6 microns). This is not the case for most polymers. The last two requirements can be met by the incorporation of additives such as high-energy absorption materials, e.g. carbon black, to improve heat absorption, and fume silica nanoparticles (talc) to aid the particle flowability with irregularly-shaped particles.

Additive manufacturing (AM), also referred to as 3D printing, involves manufacturing a part by depositing material layer-by-layer. This differs from conventional processes such as subtractive processes (i.e., milling or drilling), formative processes (i.e., casting or forging), and joining processes (i.e., welding or fastening). Quick production time, low prototyping costs, and design flexibility make 3D printing a valuable tool for both prototyping and industrial manufacturing. The three most common types of 3D printers are fused filament fabrication, stereolithography, and selective laser sintering.

Fused filament fabrication (FFF) melts a thermoplastic continuous filament and builds the object layer by layer until the print is complete. Although alternative materials exist, the two most popular filament materials are polylactic acid (PLA) and acrylonitrile butadiene styrene (ABS). FFF printers and materials are among the cheapest on the market but currently have a lower print resolution and build quality.

Stereolithography (SLA) uses a laser to polymerize photosensitive resins. Uncured liquid resin is placed in a vat where a laser is used to cure resin into solid plastic and build the object layer by layer. SLA printers have a much higher resolution than FFF printers due to the fine spot size of the laser and thus can print intricate features and complex shapes. The resins, however, are more expensive than filaments and completed prints currently require post processing with solvents to optimize the surface finish and material characteristics.

Selective laser sintering (SLS) is a powder-based layer-additive manufacturing process generally meant for rapid prototyping and rapid tooling. Laser beams either in continuous or pulse mode are used as a heat source for scanning and joining powders in predetermined sizes and shapes of layers. The geometry of the scanned layers corresponds to the various cross sections of the computer-aided design (CAD) models or stereolithography (STL) files of the object. After the first layer is scanned, a second layer of loose powder is deposited over it, and the process is repeated from bottom to top until the artifact (3D object) is complete.” (Kumar, JOM, 55(10), 43-47 (2003), incorporated by reference herein in its entirety).

SLS provides advantages for printing objects with magnetic properties that can be used for immobilizing BNCs. This is because the printing process creates porosity and a high surface area. The surrounding, unsintered powder acts as a natural support that eliminates the need for dedicated support structures. The lack of support structures allows for complex geometries that would otherwise be impossible to manufacture using alternative 3D printing methods. In addition, the nature of sintering itself creates macro and microporous volumes. During the printing process, the laser flashes thermoplastic crystalline thermoplastic powders (e.g. Polypropylene, polystyrene) between their glass transition temperature and melting temperature to generate stiff parts. By avoiding amorphous behavior with a quick laser scan speed (>100 mm/s), powders are sintered in place to form small bonds amongst themselves. The low-density powders trap air in their structures resulting in remarkable porosity and surface area in three dimensions. These pores increase the surface area for enzyme immobilization.

In recent years, industrial use of enzymes has garnered significant attention due to the wide range of potential manufacturing applications. Using enzymes in industrial processes offers several advantages over conventional chemical methods. This includes high catalytic activity, the ability to perform complex reactions, and promoting greener chemistry by reducing by-products and the need for toxic chemicals (Singh et al., Microbial enzymes: industrial progress in 21st century. 3 Biotech. 6(2):174 (2016), incorporated by reference herein in its entirety).

One of the biggest hindrances to widespread biocatalysis use in industrial production is low enzyme stability. This is further hampered by relatively harsh process conditions that can destabilize enzymes and decrease their lifespan (Mohamad et al., Biotechnology, Biotechnological Equipment 29(2):205-220 (2015), incorporated by reference herein in its entirety). Furthermore, the free enzymes used in these processes are generally lost from the system as waste products and therefore become a high operating cost. The primary solution to these issues is immobilization of enzymes onto scaffolding to enhance their operational stability and catalytic activity. Enzyme immobilization also provides a method for enzyme recovery, making biocatalytic processes more economically feasible.

Currently, biocatalytic processes for industrial production are generally carried out in batch reactors due to their simplicity and ease of operation. Despite the benefits of using batch reactors, continuous flow systems enable higher productivity and better process control (Wiles C et al., Green Chem. (14):38-54 (2012)). The rapid development of flow chemistry in biocatalytic processes has primarily been driven by a growing interest in process intensification and green chemistry. Continuous flow systems facilitate process intensification by decreasing residence times (often from hours to minutes), reducing the size of equipment required, and enabling production volume enhancement (Tamborini et al., Cell. 36(1):73-78 (2018)). From a green chemistry standpoint, these systems offer significant improvements in safety, waste generation, and energy efficiency due to heat management and mixing control (Newman and Jensen, Green Chem. (15):1456-1472 (2013)). The foregoing are incorporated by reference in their entirety

A solution to combining biocatalysis and continuous flow systems is the use of functionalized flow cells. Biocatalytic flow cells are scaffolds containing immobilized enzymes for use in reactors such as continuous stirred tank reactors (CSTRs) and packed bed reactors (PBRs). Both types of reactors are known in the art but are primarily chosen based on the type of immobilization used. With a total market value of $5.8B in 2010, immobilized enzymes are used in a diverse range of large-scale processes including high fructose corn syrup production (10⁷ tons/year), transesterification of food oils (10⁵ tons/year), biodiesel synthesis (10⁴ tons/year), and chiral resolution of alcohols and amines (10³ tons/year) (DiCosimo et al., Chem. Soc. Rev. (42):6437-6474 (2013), incorporated by reference herein in its entirety). These systems allow for improved downstream process management for enzymatic systems compared to batch reactors in terms of in-line control, enzyme reuse, and production scalability.

For the foregoing reasons, the inventions described herein provide biocatalytic systems for small-to-large scale manufacturing using BNCs in scaffolds that are shaped by 3D printing. In some embodiments, the biocatalytic systems are continuous flow.

The novel scaffolds disclosed herein may comprise cross-linked water-insoluble polymers and an approximately uniform distribution of embedded magnetic microparticles (MMP). The cross-linked polymer comprises polyvinyl alcohol (PVA) and optionally additional polymeric materials. The scaffolds may take any shape by using 3D printing. By combining natural or engineered enzymes with cofactors and cofactor recycling systems, the scaffold technology disclosed herein allows one to quickly translate innovation in biocatalysis to innovation in production. The magnetic powders are suitable for use in sequential batch reactions. For flow chemistry applications, these powders can be 3D printed as structures in a functional object or as flow cells or plate reactors. High surface areas allow one to maximize the enzyme loadings and flow can be engineered within the materials to enable biocatalysis at maximal productivity.

With the ability to immobilize any enzymes for any processes, the materials functionalized with enzymes, or enzyme systems, have applications for the production of pharmaceutical actives, agricultural actives, flavors and fragrances, fine and commodity chemicals, and food ingredients.

Self-assembled mesoporous nanoclusters comprising entrapped enzymes are highly active and robust. The technology is a powerful blend of biochemistry, nanotechnology, and bioengineering at three integrated levels of organization: Level 1 is the self-assembly of enzymes with magnetic nanoparticles (MNP) for the synthesis of magnetic mesoporous nanoclusters. This level uses a mechanism of molecular self-entrapment to immobilize and stabilize enzymes. Level 2 is the stabilization of the MNPs into other matrices. Level 3 is product conditioning and packaging for Level 1+2 delivery. The assembly of magnetic nanoparticles adsorbed to enzyme is herein also referred to as a “bionanocatalyst” (BNC).

MNPs allow for a broader range of operating conditions such as temperature, ionic strength and pH. The size and magnetization of the MNPs affect the formation and structure of the NPs, all of which have a significant impact on the activity of the entrapped enzymes. By virtue of their surprising resilience under various reaction conditions, MNPs can be used as improved enzymatic or catalytic agents where other such agents are currently used. Furthermore, they can be used in other applications where enzymes have not yet been considered or found applicable.

The BNC contains mesopores that are interstitial spaces between the magnetic nanoparticles. The enzymes are preferably embedded or immobilized within at least a portion of mesopores of the BNC. As used herein, the term “magnetic” encompasses all types of useful magnetic characteristics, including permanent magnetic, superparamagnetic, paramagnetic, ferromagnetic, and ferrimagnetic behaviors.

The magnetic nanoparticle or BNC has a size in the nanoscale, i.e., generally no more than 500 nm. As used herein, the term “size” can refer to a diameter of the magnetic nanoparticle when the magnetic nanoparticle is approximately or substantially spherical. In a case where the magnetic nanoparticle is not approximately or substantially spherical (e.g., substantially ovoid or irregular), the term “size” can refer to either the longest the dimension or an average of the three dimensions of the magnetic nanoparticle. The term “size” may also refer to an average of sizes over a population of magnetic nanoparticles (i.e., “average size”).

In different embodiments, the magnetic nanoparticle has a size of precisely, about, up to, or less than, for example, 500 nm, 400 nm, 300 nm, 200 nm, 100 nm, 50 nm, 40 nm, 30 nm, 25 nm, 20 nm, 15 nm, 10 nm, 5 nm, 4 nm, 3 nm, 2 nm, or 1 nm, or a size within a range bounded by any two of the foregoing exemplary sizes.

In the BNC, the individual magnetic nanoparticles can be considered to be primary nanoparticles (i.e., primary crystallites) having any of the sizes provided above. The aggregates of nanoparticles in a BNC are larger in size than the nanoparticles and generally have a size (i.e., secondary size) of at least about 5 nm. In different embodiments, the aggregates have a size of precisely, about, at least, above, up to, or less than, for example, 5 nm, 8 nm, 10 nm, 12 nm, 15 nm, 20 nm, 25 nm, 30 nm, 35 nm, 40 nm, 45 nm, 50 nm, 60 nm, 70 nm, 80 nm, 90 nm, 100 nm, 150 nm, 200 nm, 300 nm, 400 nm, 500 nm, 600 nm, 700 nm, or 800 nm, or a size within a range bounded by any two of the foregoing exemplary sizes.

Typically, the primary and/or aggregated magnetic nanoparticles or BNCs thereof have a distribution of sizes, i.e., they are generally dispersed in size, either narrowly or broadly dispersed. In different embodiments, any range of primary or aggregate sizes can constitute a major or minor proportion of the total range of primary or aggregate sizes. For example, in some embodiments, a particular range of primary particle sizes (for example, at least about 1, 2, 3, 5, or 10 nm and up to about 15, 20, 25, 30, 35, 40, 45, or 50 nm) or a particular range of aggregate particle sizes (for example, at least about 5, 10, 15, or 20 nm and up to about 50, 100, 150, 200, 250, or 300 nm) constitutes at least or above about 50%, 60%, 70%, 80%, 90%, 95%, 98%, 99%, or 100% of the total range of primary particle sizes. In other embodiments, a particular range of primary particle sizes (for example, less than about 1, 2, 3, 5, or 10 nm, or above about 15, 20, 25, 30, 35, 40, 45, or 50 nm) or a particular range of aggregate particle sizes (for example, less than about 20, 10, or 5 nm, or above about 25, 50, 100, 150, 200, 250, or 300 nm) constitutes no more than or less than about 50%, 40%, 30%, 20%, 10%, 5%, 2%, 1%, 0.5%, or 0.1% of the total range of primary particle sizes.

The aggregates of magnetic nanoparticles (i.e., “aggregates”) or BNCs thereof can have any degree of porosity, including a substantial lack of porosity depending upon the quantity of individual primary crystallites they are made of. In particular embodiments, the aggregates are mesoporous by containing interstitial mesopores (i.e., mesopores located between primary magnetic nanoparticles, formed by packing arrangements). The mesopores are generally at least 2 nm and up to 50 nm in size. In different embodiments, the mesopores can have a pore size of precisely or about, for example, 2, 3, 4, 5, 10, 12, 15, 20, 25, 30, 35, 40, 45, or 50 nm, or a pore size within a range bounded by any two of the foregoing exemplary pore sizes. Similar to the case of particle sizes, the mesopores typically have a distribution of sizes, i.e., they are generally dispersed in size, either narrowly or broadly dispersed. In different embodiments, any range of mesopore sizes can constitute a major or minor proportion of the total range of mesopore sizes or of the total pore volume. For example, in some embodiments, a particular range of mesopore sizes (for example, at least about 2, 3, or 5, and up to 8, 10, 15, 20, 25, or 30 nm) constitutes at least or above about 50%, 60%, 70%, 80%, 90%, 95%, 98%, 99%, or 100% of the total range of mesopore sizes or of the total pore volume. In other embodiments, a particular range of mesopore sizes (for example, less than about 2, 3, 4, or 5 nm, or above about 10, 15, 20, 25, 30, 35, 40, 45, or 50 nm) constitutes no more than or less than about 50%, 40%, 30%, 20%, 10%, 5%, 2%, 1%, 0.5%, or 0.1% of the total range of mesopore sizes or of the total pore volume.

The magnetic nanoparticles can have any of the compositions known in the art. In some embodiments, the magnetic nanoparticles are or include a zerovalent metallic portion that is magnetic. Some examples of such zerovalent metals include cobalt, nickel, and iron, and their mixtures and alloys. In other embodiments, the magnetic nanoparticles are or include an oxide of a magnetic metal, such as an oxide of cobalt, nickel, or iron, or a mixture thereof. In some embodiments, the magnetic nanoparticles possess distinct core and surface portions. For example, the magnetic nanoparticles may have a core portion composed of elemental iron, cobalt, or nickel and a surface portion composed of a passivating layer, such as a metal oxide or a noble metal coating, such as a layer of gold, platinum, palladium, or silver. In other embodiments, metal oxide magnetic nanoparticles or aggregates thereof are coated with a layer of a noble metal coating. The noble metal coating may, for example, reduce the number of charges on the magnetic nanoparticle surface, which may beneficially increase dispersibility in solution and better control the size of the BNCs. The noble metal coating protects the magnetic nanoparticles against oxidation, solubilization by leaching or by chelation when chelating organic acids, such as citrate, malonate, or tartrate are used in the biochemical reactions or processes. The passivating layer can have any suitable thickness, and particularly, at least, up to, or less than, about for example, 0.1 nm, 0.2 nm, 0.3 nm, 0.4 nm, 0.5 nm, 0.6 nm, 0.7 nm, 0.8 nm, 0.9 nm, 1 nm, 2 nm, 3 nm, 4 nm, 5 nm, 6 nm, 7 nm, 8 nm, 9 nm, or 10 nm, or a thickness in a range bounded by any two of these values.

Magnetic materials useful for the invention are well-known in the art. Non-limiting examples comprise ferromagnetic and ferromagnetic materials including ores such as iron ore (magnetite or lodestone), cobalt, and nickel. In other embodiments, rare earth magnets are used. Non-limiting examples include neodymium, gadolinium, sysprosium, samarium-cobalt, neodymium-iron-boron, and the like. In yet further embodiments, the magnets comprise composite materials. Non-limiting examples include ceramic, ferrite, and alnico magnets. In preferred embodiments, the magnetic nanoparticles have an iron oxide composition. The iron oxide composition can be any of the magnetic or superparamagnetic iron oxide compositions known in the art, e.g., magnetite (Fe₃O₄), hematite (α-Fe₂O₃), maghemite (γ-Fe₂O₃), or a spinel ferrite according to the formula AB₂O₄, wherein A is a divalent metal (e.g., Xn²⁺, Ni²⁺, Mn²⁺, Co²⁺, Ba²⁺, Sr²⁺, or combination thereof) and B is a trivalent metal (e.g., Fe³⁺, Cr³⁺, or combination thereof).

The individual magnetic nanoparticles or aggregates thereof or BNCs thereof possess any suitable degree of magnetism. For example, the magnetic nanoparticles, BNCs, or BNC scaffold assemblies can possess a saturated magnetization (Ms) of at least or up to about 5, 10, 15, 20, 25, 30, 40, 45, 50, 60, 70, 80, 90, or 100 emu/g. The magnetic nanoparticles, BNCs, or BNC-scaffold assemblies preferably possess a permanent magnetization (Mr) of no more than (i.e., up to) or less than 5 emu/g, and more preferably, up to or less than 4 emu/g, 3 emu/g, 2 emu/g, 1 emu/g, 0.5 emu/g, or 0.1 emu/g. The surface magnetic field of the magnetic nanoparticles, BNCs, or BNC-scaffold assemblies can be about or at least, for example, about 0.5, 1, 5, 10, 50, 100, 200, 300, 400, 500, 600, 700, 800, 900, or 1000 Gauss (G), or a magnetic field within a range bounded by any two of the foregoing values. If microparticles are included, the microparticles may also possess any of the above magnetic strengths.

The magnetic nanoparticles or aggregates thereof can be made to adsorb a suitable amount of enzyme, up to or below a saturation level, depending on the application, to produce the resulting BNC. In different embodiments, the magnetic nanoparticles or aggregates thereof may adsorb about, at least, up to, or less than, for example, 1, 5, 10, 15, 20, 25, or 30 pmol/m² of enzyme. Alternatively, the magnetic nanoparticles or aggregates thereof may adsorb an amount of enzyme that is about, at least, up to, or less than, for example, about 10%, 20%, 30%, 40%, 50%, 60%, 70%, 80%, 90%, or 100% of a saturation level.

The magnetic nanoparticles or aggregates thereof or BNCs thereof possess any suitable pore volume. For example, the magnetic nanoparticles or aggregates thereof can possess a pore volume of about, at least, up to, or less than, for example, about 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1 cm³/g, or a pore volume within a range bounded by any two of the foregoing values.

The magnetic nanoparticles or aggregates thereof or BNCs thereof possess any suitable specific surface area. For example, the magnetic nanoparticles or aggregates thereof can have a specific surface area of about, at least, up to, or less than, for example, about 50, 60, 70, 80, 90, 100, 110, 120, 130, 140, 150, 160, 170, 180, 190, or 200 m²/g.

MNPs, their structures, organizations, suitable enzymes, and uses are described in WO2012122437 and WO2014055853, incorporated by reference herein in their entirety.

Some embodiments of the invention comprise hydrolases. Hydrolases catalyze the hydrolysis of many types of chemical bonds by using water as a substrate. The substrates typically have hydrogen and hydroxyl groups at the site of the broken bonds. Hydrolases are classified as EC 3 in the EC number classification of enzymes. Hydrolases can be further classified into several subclasses, based upon the bonds they act upon. Exemplary hydrolases and the bonds they hydrolyze include EC 3.1: ester bonds (esterases: nucleases, phosphodiesterases, lipase, phosphatase), EC 3.2: sugars (DNA glycosylases, glycoside hydrolase), EC 3.3: ether bonds, EC 3.4: peptide bonds (Proteases/peptidases), EC 3.5: carbon-nitrogen bonds, other than peptide bonds, EC 3.6 acid anhydrides (acid anhydride hydrolases, including helicases and GTPase), EC 3.7 carbon-carbon bonds, EC 3.8 halide bonds, EC 3.9: phosphorus-nitrogen bonds, EC 3.10: sulphur-nitrogen bonds, EC 3.11: carbon-phosphorus bonds, EC 3.12: sulfur-sulfur bonds, and EC 3.13: carbon-sulfur bonds.

In some preferred embodiments, the hydrolase is a glycoside hydrolase. These enzymes have a variety of uses including degradation of plant materials (e.g. cellulases for degrading cellulose to glucose that are used for ethanol production), food manufacturing (e.g. sugar inversion, maltodextrin production), and paper production (removing hemicelluloses from paper pulp).

In some preferred embodiments, the hydrolase is lipolase 100L (EC 3.1.1.3). It is used to synthesize pregabalin (marketed as by Pfizer as Lyrica®), an anticonvulsant drug used for neuropathic pain, anxiety disorders, and epilepsy. These conditions affect about 1% of the world's population. Lipolase 100L was found to reduce the required starting material by 39% and cut the waste per unit by 80%.

In some preferred embodiments, the hydrolase is a gamma-lactamase (e.g. EC 3.1.5.49). It is used to make Vince lactam, an intermediate for abacavir production (an antiretroviral drug for treating HIV/AIDS). It was found that changing from a stoichiometric process to a catalytic flow process reduced the number of unit operations from 17 to 12 and reduced the waste by 35%. Additionally, the use of the toxic substance cyanogen chloride is minimized.

In some preferred embodiments, the hydrolase is a Lactase (e.g. EC 3.2.1.108). These enzymes break apart lactose in milk into simple sugars to produce lactose-free milk. This important product serves approximately 15% of the world population that is lactose intolerant.

In some preferred embodiments, the hydrolase is a penicillin amidase (e.g. EC 3.5.1.11). These enzymes split penicillin into a carboxylate and 6-aminopenicillanate (6-APA). 6-APA is the core structure in natural and synthetic penicillin derivatives. These enzymes are used to produce semisynthetic penicillins tailored to fight specific infections.

In some preferred embodiments, the hydrolase is a nitralase (e.g. EC 3.5.5.1). These enzymes split nitriles into carboxyl groups. A nitralase is used to manufacture atorvastatin (marketed by Pfizer as Lipitor®). It catalyzes the reaction of meso-3-hydroxyglutaronitrile to ethyl (R)-4-cyano-3-hydroxybutyrate, the latter of which form the core of atorvastatin.

Hydrolases are discussed in the following references, incorporated herein by reference in their entirety: Anastas, P. T. Handbook of Green Chemistry. Wiley-VCH-Verlag, 2009; Dunn, Peter J., Andrew Wells, and Michael T. Williams, eds. Green chemistry in the pharmaceutical industry. John Wiley & Sons, 2010.; Martinez et al., Curr. Topics Med. Chem. 13(12):1470-90 (2010); Wells et al., Organic Process Res. Dev. 16(12):1986-1993 (2012).

In some embodiments, the invention provides hydrogen peroxide producing (HPP) enzymes. In certain embodiments, the HPP enzymes are oxidases that may be of the EX 1.1.3 subgenus. In particular embodiments, the oxidase may be EC 1.1.3.3 (malate oxidase), EC 1.1.3.4 (glucose oxidase), EC 1.1.3.5 (hexose oxidase), EC 1.1.3.6 (cholesterol oxidase), EC 1.1.3.7 (aryl-alcohol oxidase), EC 1.1.3.8 (L-gulonolactone oxidase), EC 1.1.3.9 (galactose oxidase), EC 1.1.3.10 (pyranose oxidase), EC 1.1.3.11 (L-sorbose oxidase), EC 1.1.3.12 (pyridoxine 4-oxidase), EC 1.1.3.13 (alcohol oxidase), EC 1.1.3.14 (catechol oxidase), EC 1.1.3.15 (2-hydroxy acid oxidase), EC 1.1.3.16 (ecdysone oxidase), EC 1.1.3.17 (choline oxidase), EC 1.1.3.18 (secondary-alcohol oxidase), EC 1.1.3.19 (4-hydroxymandelate oxidase), EC 1.1.3.20 (long-chain alcohol oxidase), EC 1.1.3.21 (glycerol-3-phosphate oxidase), EC 1.1.3.22, EC 1.1.3.23 (thiamine oxidase), EC 1.1.3.24 (L-galactonolactone oxidase), EC 1.1.3.25, EC 1.1.3.26, EC 1.1.3.27 (hydroxyphytanate oxidase), EC 1.1.3.28 (nucleoside oxidase), EC 1.1.3.29 (Nacylhexosamine oxidase), EC 1.1.3.30 (polyvinyl alcohol oxidase), EC 1.1.3.31, EC 1.1.3.32, EC 1.1.3.33, EC 1.1.3.34, EC 1.1.3.35, EC 1.1.3.36, EC 1.1.3.37 D-arabinono-1,4-lactone oxidase), EC 1.1.3.38 (vanillyl alcohol oxidase), EC 1.1.3.39 (nucleoside oxidase, H₂O₂ forming), EC 1.1.3.40 (D-mannitol oxidase), or EC 1.1.3.41 (xylitol oxidase).

Some embodiments of the invention may comprise hydroxylases. Hydroxylation is a chemical process that introduces a hydroxyl group (—OH) into an organic compound. Hydroxylation is the first step in the oxidative degradation of organic compounds in air. Hydroxylation plays a role in detoxification by converting lipophilic compounds into hydrophilic products that are more readily excreted. Some drugs (e.g. steroids) are activated or deactivated by hydroxylation. Hydroxylases are well-known in the art. Exemplary hydroxylases include proline hydroxylases, lysine hydroxylases, and tyrosine hydroxylases.

Some embodiments of the invention comprise Nitrilases (NIT). They are hydrolyzing enzymes (EC 3.5.5.1) that catalyze the hydrolysis of nitriles into chiral carboxylic acids with high enantiopurity and ammonia. NIT activity may be measured by monitoring the conversion of mandelonitirile into a (R)-mandelic acid. This results in a pH drop that may be monitored spectrophotometrically. Nitrilases are used to produce nicotinic acid, also known as vitamin B3 or niacin, from 3-cyanopyridine. Nicotinic acid is a nutritional supplement in foods and a pharmaceutical intermediate. Exemplary industrial uses are discussed in Gong et al., Microbial Cell Factories, 11(1), 142 (2012), incorporated herein by reference herein in its entirety.

Some embodiments of the invention comprise hydratases. They are enzymes that catalyze the addition or removal of the elements of water. Hydratases, also known as hydrolases or hydrases, may catalyze the hydration or dehydration of C—O linkages.

Some embodiments of the invention comprise oxidoreductases. These enzymes catalyze the transfer of electrons from one molecule to another. This involves the transfer of H and O atoms or electrons from one substance to another. They typically utilize NADP or NAD+ as cofactors.

In some preferred embodiments of the invention, Oxidoreductases are used for the decomposition of pollutants such as polychlorinated biphenyls and phenolic compounds, the degradation of coal, and the enhancement of the fermentation of wood hydrolysates. The invention further includes their use in biosensors and disease diagnosis.

In some preferred embodiments, the oxidoreductase is a dehydrogenase (DHO). This group of oxidoreductases oxidizes a substrate by a reduction reaction that transfers one or more hydrides (H—) to an electron acceptor, usually NAD+/NADP+ or a flavin coenzyme such as FAD or FMN. Exemplary dehydrogenases include aldehyde dehydrogenase, acetaldehyde dehydrogenase, alcohol dehydrogenase, glutamate dehydrogenase, lactate dehydrogenase, pyruvate dehydrogenase, glucose-6-phosphate dehydrogenase, glyceraldehyde-3-phosphate dehydrogenase, sorbitol dehydrogenase, isocitrate dehydrogenase, alpha-ketoglutarate dehydrogenase, succinate dehydrogenase, and malate dehydrogenase.

In some preferred embodiments, the oxidoreductase is a ketoreductase (EC 1.1.1.184), an oxidoreductase used to make atorvastatin (marketed by Pfizer as Lipitor®). This biocatalytic process is commercially important because it substantially reduces starting materials, limits the use of organic solvents, and increases the biodegradability of the waste streams.

In some preferred embodiments, the oxidoreductase is a glucose dehydrogenase (e.g. EC 1.1.99.10). They are used by pharmaceutical companies to recycle cofactors used in drug production. They catalyze the transformation of glucose into gluconate. NADP+ is reduced to NADPH. This is used in Avastan production.

Cytochrome P450 (referred to as P450 or CYP) are of the E.C. 1.14 class of enzymes. (Br. J. Pharmacol. 158(Suppl 1): S215-S217 (2009), incorporated by reference herein in its entirety). They constitute a family of monooxygenases involved in the biotransformation of drugs, xenobiotics, alkanes, terpenes, and aromatic compounds. They also participate in the metabolism of chemical carcinogens and the biosynthesis of physiologically relevant compounds such as steroids, fatty acids, eicosanoids, fat-soluble vitamins, and bile acids. Furthermore, they are also involved in the degradation of xenobiotics in the environment such pesticides and other industrial organic contaminants. They function by incorporating one hydroxyl group into substrates found in many metabolic pathways. In this reaction, dioxygen is reduced to one hydroxyl group and one H₂O molecule by the concomitant oxidation of a cofactor such as NAD(P)H.

In some preferred embodiments, the oxidoreductase is P450 (EC 1.14.14.1). It is used in the pharmaceutical industry for difficult oxidations. P450 reduces the cost, inconsistency, and inefficiency associated with natural cofactors (e.g., NADPH/NADP+). There are several advantages of using drug metabolites as active ingredients because they can show superior properties compared to the original drugs. This includes improved pharmacodynamics, improved pharmacokinetics, lower probability of drug-drug interactions, less variable pharmacokinetics and/or pharmacodynamics, improved overall safety profile and improved physicochemical properties.

In some embodiments, the P450 monooxygenase comprises a P450 sequence that is mammalian. In other embodiments, the P450 monooxygenase comprises a P450 sequence that is human. In other embodiments, the P450 monooxygenase comprises CYP1A1, CYP1A2, CYP1B1, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3A4, CYP3A5, CYP3A7, CYP3A43, CYP4A11, CYP4A22, CYP4B1, CYP4F2, CYP4F3, CYP4F8, CYP4F11, CYP4F12, CYP4F22, CYP4V2, CYP4X1, CYP4Z1, CYP5A1, CYP7A1, CYP7B1, CYP8A1, CYP8B1, CYP11A1, CYP11B1, CYP11B2, CYP17A1, CYP19A1, CYP20A1, CYP21A2, CYP24A1, CYP26A1, CYP26B1, CYP26C1, CYP27A1, CYP27B1, CYP27C1, CYP39A1, CYP46A1, or CYP51A1.

In some embodiments, the P450 monooxygenase comprises a P450 sequence that is of an origin selected from the group consisting of primate, mouse, rat, dog, cat, horse, cow, sheep, and goat. In other embodiments, the P450 monooxygenase comprises a P450 sequence that is of an origin selected from the group consisting of insect, fish, fungus, yeast, protozoan, and plant.

In some embodiments, the P450 is in a soluble form such as the BM3 P450 from Bacillus megaterium. See, e.g., SEQ ID NO:1. In other embodiments, the BM3 P450 has one or more variant amino acids from the wild-type. In other embodiments, the P450 has at least a 90% sequence identity to SEQ ID NO:1.

In some embodiments of the invention, the cofactor is nicotinamide adenine dinucleotide+hydrogen (NADH), nicotinamide adenine dinucleotide phosphate+hydrogen (NADPH), Flavin adenine dinucleotide+hydrogen (FADH), or glutathione.

In some preferred embodiments, the oxidoreductase is a catalase such as EC 1.11.1.6. It is used in the food industry for removing hydrogen peroxide from milk prior to cheese production and for producing acidity regulators such as gluconic acid. Catalase is also used in the textile industry for removing hydrogen peroxide from fabrics.

In some preferred embodiments, the oxidoreductase is a glucose oxidase (e.g. Notatin, EC 1.1.3.4). It catalyzes the oxidation of glucose to hydrogen peroxide and D-glucono-δ-lactone. It is used, for example, to generate hydrogen peroxide as an oxidizing agent for hydrogen peroxide consuming enzymes such as peroxidase.

In some embodiments, the invention encompasses Free Radical Producing (FRP) enzymes. In some embodiments, the FRP is a peroxidase. Peroxidases are widely found in biological systems and form a subset of oxidoreductases that reduce hydrogen peroxide (H₂O₂) to water in order to oxidize a large variety of aromatic compounds ranging from phenol to aromatic amines. Peroxidases are very potent enzymes yet notoriously difficult to deploy in industrial settings due to strong inhibition in presence of excess peroxide. The invention provides increased reaction turnover and reduced inhibition. Thus, enzymes such as Horseradish Peroxidase (HRP) may be used at industrial scales.

Peroxidases belong to the sub-genus EC 1.11.1. In certain embodiments, the EC 1.11.1 enzyme is The EC 1.11.1 enzyme can be more specifically, for example, EC 1.11.1.1 (NADH peroxidase), EC 1.11.1.2 (NADPH peroxidase), EC 1.11.1.3 (fatty acid peroxidase), EC 1.11.1.4, EC 1.11.1.5 (cytochrome-c peroxidase), EC 1.11.1.6 (catalase), EC 1.11.1.7 (peroxidase), EC 1.11.1.8 (iodide peroxidase), EC 1.11.1.9 (glutathione peroxidase), EC 1.11.1.10 (chloride peroxidase), EC 1.11.1.11 (L-ascorbate peroxidase), EC 1.11.1.12 (phospholipid-hydroperoxide glutathione peroxidase), EC 1.11.1.13 (manganese peroxidase), EC 1.11.1.14 (diarylpropane peroxidase), or EC 1.11.1.15 (peroxiredoxin).

Horseradish peroxidase (EC 1.11.1.7) is a heme-containing oxidoreductase enzyme found in the roots of the horseradish plant A. rusticana. It is commonly used as a biochemical signal amplifier and tracer, as it usually acts on a chromogenic substrate together with hydrogen peroxide to produce a brightly colored product complex. It improves spectrophotometric detectability of target molecules. This characteristic of horseradish peroxidase (HRP) has been applied to permeability studies of rodent nervous system capillaries. In some embodiments of the invention, HRP is used as part of a possible remediation strategy of phenolic wastewaters due to its ability to degrade various aromatic compounds. See Duan et al., ChemPhysChem, 15(5), 974-980 (2014), incorporated by reference herein in its entirety.

In other embodiments, the peroxidase may also be further specified by function, e.g., a lignin peroxidase, manganese peroxidase, or versatile peroxidase. The peroxidase may also be specified as a fungal, microbial, animal, or plant peroxidase. The peroxidase may also be specified as a class I, class II, or class III peroxidase. The peroxidase may also be specified as a myeloperoxidase (MPO), eosinophil peroxidase (EPO), lactoperoxidase (LP), thyroid peroxidase (TPO), prostaglandin H synthase (PGHS), glutathione peroxidase, haloperoxidase, catalase, cytochrome c peroxidase, horseradish peroxidase, peanut peroxidase, soybean peroxidase, turnip peroxidase, tobacco peroxidase, tomato peroxidase, barley peroxidase, or peroxidasin. In particular embodiments, the peroxidase is horseradish peroxidase.

The lactoperoxidase/glucose oxidase (LP/GOX) antimicrobial system occurs naturally in bodily fluids such as milk, saliva, tears, and mucous (Bosch et al., J. Applied Microbiol., 89(2), 215-24 (2000)). This system utilizes thiocyanate (SCN—) and iodide (I—), two naturally occurring compounds that are harmless to mammals and higher organisms (Welk et al. Archives of Oral Biology, 2587 (2011)). LP catalyzes the oxidation of thiocyanate and iodide ions into hypothiocyanite (OSCN—) and hypoiodite (OI—), respectively, in the presence of hydrogen peroxide (H₂O₂). The H₂O₂ in this system is provided by the activity of GOX on β-D-glucose in the presence of oxygen. These free radical compounds, in turn, oxidize sulfhydryl groups in the cell membranes of microbes (Purdy, Tenovuo et al. Infection and Immunity, 39(3), 1187 (1983); Bosch et al., J. Applied Microbiol., 89(2), 215-24 (2000), leading to impairment of membrane permeability (Wan, Wang et al. Biochemistry Journal, 362, 355-362 (2001)) and ultimately microbial cell death.

Some embodiments of the invention comprise transferases. “Transferase” refers to a class of enzymes that transfer specific functional groups from one molecule to another. Examples of groups transferred include methyl groups and glycosyl groups. Transferases are used for treating substances such as chemical carcinogens and environmental pollutants. Additionally, they are used to fight or neutralize toxic chemicals and metabolites found in the human body.

In some preferred embodiments, the transferase is a transaminase. A transaminase or an aminotransferase catalyzes a reaction between an amino acid and an α-keto acid. They are important in the synthesis of amino acids. In transamination, the NH₂ group on one molecule is exchanged with the ═O from another group (e.g. a keto group) on the other molecule.

In more preferred embodiments, the transaminase is w-transaminases (EC 2.6.1.18). It is used, among other things, to synthesize sitagliptin (marketed by Merck and Co. as Januvia®, an antidiabetic drug). Engineered ω-transaminases were found to improve biocatalytic activity by, for example, 25,000 fold, resulting in a 13% overall increase in sitagliptin yield and 19% reduction in overall process waste.

Due to their high stereoselectivity for substrates and stereospecificity for products, ω-transaminases can be utilized to make unnatural amino acids and optically pure chiral amines or keto acids (Mathew & Yun, ACS Catalysis 2(6), 993-1001 (2012)). ω-Transaminases also have applications in biocatalytic chiral resolution of active pharmaceutical intermediates, simplifying the process over conventional chemical methods. (Schatzle et al., Anal. Chem. 81(19):8244-48 (2009).) The foregoing are incorporated by reference in their entirety.

In some preferred embodiments, the transferase is a thymidylate synthetase (e.g. EC 2.1.1.45). These enzymes are used for manufacturing sugar nucleotides and oligosaccharides. They catalyze, for example, the following reaction:

5,10-methylenetetrahydrofolate+dUMP [symbol] dihydrofolate+dTMP.

In some preferred embodiments, the transferase is a glutathione S-transferase (e.g. EC 2.5.1.18). These enzymes catalyze glutathione into other tripeptides. They are used in the food industry as oxidizing agents as well as in the pharmaceutical industry to make anti-aging drugs and skin formulations.

In some preferred embodiments, the transferase is a glucokinase (e.g. EC 2.7.1.2). These enzymes facilitate the phosphorylation of glucose to glucose-6-phosphate. They are used in the food industry to reduce the glucose concentration in their production streams and as in the pharmaceutical industry to make diabetes drugs.

In some preferred embodiments, the transferase is a riboflavin kinase (e.g. EC 2.7.1.26). In a more preferred embodiment, a riboflavin kinase is used to produce flavin mononucleotide (FMN) in the food industry. FMN is an orange-red food color additive and an agent that breaks down excess riboflavin (vitamin B2). Riboflavin kinase catalyzes, for example, the following reaction:

ATP+riboflavin [symbol] ADP+Flavin mononucleotide (FMN).

Some embodiments of the invention comprise ene reductases (EREDS). These enzymes catalyze alkene reduction in an NAD(P)H-dependent manner. Examples of ene reductases include The FMN-containing Old Yellow Enzyme (OYE) family of oxidoreductases (EC 1.6.99), clostridial enoate reductases (EnoRs, C 1.3.1.31), flavin-independent medium chain dehydrogenase/reductases (MDR; EC 1.3.1), short chain dehydrogenase/reductases (SDR; EC 1.1.1.207-8), leukotriene B4 dehydrogenase (LTD), quinone (QOR), progesterone 5b-reductase, rat pulegone reductase (PGR), tobacco double bond reductase (NtDBR), Cyanobacterial OYEs, LacER from Lactobacillus casei, Achr-OYE4 from Achromobacter sp. JA81, and Yeast OYEs.

Some embodiments of the invention comprise imine reductases (IREDS). Imine reductases (IRED) catalyze the synthesis of optically pure secondary cyclic amines. They may convert a ketone or aldehyde substrate and a primary or secondary amine substrate to form a secondary or tertiary amine product compound. Exemplary IREDs are those from Paenibacillus elgii B69, Streptomyces ipomoeae 91-03, Pseudomonas putida KT2440, and Acetobacterium woodii IREDs are discussed in detail in Int'l Pub. No. WO2013170050, incorporated by reference herein in its entirety.

In some embodiments of the invention, the enzymes are lyases. They catalyze elimination reactions in which a group of atoms is removed from a substrate by a process other than hydrolysis or oxidation. A new double bond or ring structure often results. Seven subclasses of lyases exist. In preferred embodiments, pectin lyase is used to degrade highly esterified pectins (e.g. in fruits) into small molecules. Other preferred embodiments of the invention comprise oxynitrilases (also referred to as mandelonitrile lyase or aliphatic (R)-hydroxynitrile lyase). They cleave mandelonitrile into hydrogen cyanide+benzaldehyde.

In a preferred embodiment, the lyase is a hydroxynitrile lyase (e.g. EC 4.1.2, a mutation of a Prunus amygdalus lyase). Hydroxynitrile lyases catalyze the formation of cyanohydrins which can serve as versatile building blocks for a broad range of chemical and enzymatic reactions. They are used to improve enzyme throughput and stability at a lower pH and is used for producing clopidogrel (Plavix®). The reaction process is described in Glieder et al., Chem. Int. Ed. 42:4815 (2003), incorporated by reference herein in its entirety.

In another preferred embodiment, the lyase is 2-deoxy-D-ribose phosphate aldolase (DERA, EC 4.1.2.4). It is used for forming statin side chains, e.g. in Lipitor production.

In another preferred embodiment, the lyase is (R)-mandelonitrile lyase (HNL, EC 4.1.2.10). It is used to synthesize Threo-3-Aryl-2,3-dihydroxypropanoic acid, a precursor cyanohydrin used to produce Diltiazem. Diltiazem is a cardiac drug that treats high blood pressure and chest pain (angina). Lowering blood pressure reduces the risk of strokes and heart attacks. It is a calcium channel blocker. Ditiazem and its production are described in Dadashipour and Asano, ACS Catal. 1:1121-49 (2011) and Aehle W. 2008. Enzymes in Industry, Weiley-VCH Verlag, GmbH Weinheim, both of which are incorporated by reference in their entirety.

In another preferred embodiment, the lyase is nitrile hydratase (EC 4.2.1). It is used commercially to convert 3-cyanopyridine to nicotinamide (vitamin B3, niacinamide). It is also used in the preparation of levetiracetam, the active pharmaceutical ingredient in Keppra®.

In another preferred embodiment, the lyase is a Phenyl Phosphate Carboxylase. They are used, e.g., for phosphorylating phenol at room temperature and under sub-atmospheric CO₂ pressure. These enzymes catalyze the synthesis of 4-OH benzoic acid from phenol and CO₂ with 100% selectivity. 4-OH benzoic acid is used in the preparation of its esters. In more preferred embodiments, the enzymes are used for producing parabens that are used as preservatives in cosmetics and opthalmic solutions.

In some embodiments of the invention, the enzyme is a carbonic anhydrase (e.g. EC 4.2.1.1). Carbonic anhydrases are ubiquitous metalloenzymes present in every organism. They are among the most efficient enzymes known and serve multiple physiological roles including CO₂ exchange, pH regulation, and HCO₃⁻ secretion. Carbonic anhydrase also has potential industrial applications in CO₂ sequestration and calcite production. See Lindskog & Silverman, (2000), The catalytic mechanism of mammalian carbonic anhydrases EXS 90:175-195 (W. R. Chegwidden et al. eds. 2000); In The Carbonic Anhydrases: New Horizons 7^{th} Edition pp. 175-95 (W. R. Chegwidden et al. eds. 2000); McCall et al., J. Nutrition 130:1455-1458 (2000); Boone et al., Int'l J. Chem. Engineering Volume 2013: 22-27 (2013). The foregoing are incorporated by reference in their entirety.

In some embodiments of the invention, the enzyme is an isomerase. Isomerases catalyze molecular isomerizations, i.e. reactions that convert one isomer to another. They can facilitate intramolecular rearrangements in which bonds are broken and formed or they can catalyze conformational changes. Isomerases are well known in the art.

In preferred embodiments, isomerases are used in sugar manufacturing. In more preferred embodiments, the isomerase is Glucose isomerase, EC 5.3.1.18. In other embodiments, the glucose isomerase is produced by Actinoplanes missouriensis, Bacillus coagulans or a Streptomyces species. Glucose isomerase converts D-xylose and D-glucose to D-xylulose and D-fructose, important reactions in the production of high-fructose corn syrup and in the biofuels sector.

In another preferred embodiment, the isomerase is Maleate cis-trans isomerase (EC 5.2.1.1). It catalyzes the conversion of maleic acid into fumaric acid. Fumaric acid is important for the biocatalytic production of L-aspartic acid, L-malic acid, polyester resins, food and beverage additives, and mordant for dyes.

In another preferred embodiment, the isomerase is linoleate cis-trans isomerase (EC 5.2.1.5). It catalyzes the isomerization of conjugated linoleic acid (CLA). CLA has been reported to have numerous potential health benefits for treating obesity, diabetes, cancer, inflammation, and artherogenesis. Different isomers of CLA may exert differential physiological effects. Thus, the enzyme is used to prepare single isomers.

In another preferred embodiment of the invention, the isomerase is triosephosphate isomerase (EC 5.3.1.1). It catalyzes the interconversion of D-glyceraldehyde 3-phosphate and dihydroxyacetone phosphate. In combination with transketolases or aldolases, triosephosphate isomerase is used in the stereoselective multienzyme synthesis of various sugars or sugar analogs. A preferred embodiment is the one-pot enzymatic preparation of D-xylulose 5-phosphate. This synthesis starts with the retro-aldol cleavage of fructose 1,6-biphosphate by D-fructose 1,6-biphosphate aldolase (EC 4.1.2.13). The following racemization, triosephosphate isomerase facilitates the generation of two equivalents of D-glyceraldehyde 3-phosphate that is converted into xylulose 5-phosphate by transketolase (EC 2.2.1.1)

In other embodiments of the invention, the enzyme is a Ligase. These enzymes catalyze the formation of covalent bonds joining two molecules together, coupled with the hydrolysis of a nucleoside-triphosphate. Ligases are well-known in the art and are commonly used for recombinant nucleic acid applications. In a preferred embodiment, the DNA ligase is EC 6.5.1.1.

In a preferred embodiment, the ligase is Acetyl-CoA Carboxylase (EC 6.4.1.2, ACC). ACC has a role at the junction of the lipid synthesis and oxidation pathways. It is used with the inventions disclosed herein for clinical purposes such as the production of antibiotics, diabetes therapies, obesity, and other manifestations of metabolic syndrome.

In another preferred embodiment, the ligase is Propionyl-CoA Carboxylase (PCC, EC 6.4.1.3). It catalyzes the biotin-dependent carboxylation of propionyl-CoA to produce D-methylmalonyl-CoA in the mitochondrial matrix. Methylmalyl-CoA is an important intermediate in the biosynthesis of many organic compounds as well as the process of carbon assimilation.

In some embodiments, the methods described herein use recombinant cells that express the enzymes used in the invention. Recombinant DNA technology is known in the art. In some embodiments, cells are transformed with expression vectors such as plasmids that express the enzymes. In other embodiments, the vectors have one or more genetic signals, e.g., for transcriptional initiation, transcriptional termination, translational initiation and translational termination. Here, nucleic acids encoding the enzymes may be cloned in a vector so that they are expressed when properly transformed into a suitable host organism. Suitable host cells may be derived from bacteria, fungi, plants, or animals as is well-known in the art.

Although BNCs (Level 1) provide the bulk of enzyme immobilization capability, they are sometimes too small to be easily captured by standard-strength magnets. Thus, sub-micrometric magnetic materials (Level 2) are used to provide bulk magnetization and added stability to Level 1. Commercially available free magnetite powder, with particle sizes ranging from 50-500 nm, is highly hydrophilic and tends to stick to plastic and metallic surfaces, which, over time, reduces the effective amount of enzyme in a given reactor system. In addition, powdered magnetite is extremely dense, thus driving up shipping costs. It is also rather expensive—especially at particle sizes finer than 100 nm. To overcome these limitations, low-density hybrid materials consisting of magnetite, non-water-soluble cross-linked polymers such as poly(vinylalcohol) (PVA) and carboxymethylcellulose (CMC), have been developed. These materials are formed by freeze-casting and freeze-drying water-soluble polymers followed by cross-linking. These materials have reduced adhesion to external surfaces, require less magnetite, and achieve Level 1 capture that is at least comparable to that of pure magnetite powder.

In one embodiment, the continuous macroporous scaffold has a cross-linked polymeric composition. The polymeric composition can be any of the solid organic, inorganic, or hybrid organic-inorganic polymer compositions known in the art, and may be synthetic or a biopolymer that acts as a binder. Preferably, the polymeric macroporous scaffold does not dissolve or degrade in water or other medium in which the hierarchical catalyst is intended to be used. Some examples of synthetic organic polymers include the vinyl addition polymers (e.g., polyethylene, polypropylene, polystyrene, polyacrylic acid or polyacrylate salt, polymethacrylic acid or polymethacrylate salt, poly(methylmethacrylate), polyvinyl acetate, polyvinyl alcohol, and the like), fluoropolymers (e.g., polyvinylfluoride, polyvinylidenefluoride, polytetrafluoroethylene, and the like), the epoxides (e.g., phenolic resins, resorcinol—formaldehyde resins), the polyamides, the polyurethanes, the polyesters, the polyimides, the polybenzimidazoles, and copolymers thereof. Some examples of biopolymers include the polysaccharides (e.g., cellulose, hemicellulose, xylan, chitosan, inulin, dextran, agarose, and alginic acid), polylactic acid, and polyglycolic acid. In the particular case of cellulose, the cellulose may be microbial- or algae-derived cellulose. Some examples of inorganic or hybrid organic-inorganic polymers include the polysiloxanes (e.g., as prepared by sol gel synthesis, such as polydimethylsiloxane) and polyphosphazenes. In some embodiments, any one or more classes or specific types of polymer compositions provided above are excluded as macroporous scaffolds.

In order that the invention described herein may be more fully understood, the following examples are set forth. It should be understood that these examples are for illustrative purposes only and are not to be construed as limiting this invention in any manner.

### Example 1—Thermoplastics and Magnetite Blends for 3D Printing by Melting and Extrusion

Materials: Polyamide 12 (Nylon 12, Cat No. AdSint PA12) and polystyrene (Cat. No. Coathylene PS Sint) were purchased from Advanc3D Materials GmbH (Hamburg, Germany). Polypropylene (Cat. No. Polyaxis PD 2000) sample was obtained from A. Schulman (Fairlawn, Ohio). Iron oxide (Cat. No. QM-D50/4 Magnetite) with a particle size smaller than 10 μm was obtained from Reade Advanced Materials (East Providence, R.I.) and iron (II, III) oxide particle size smaller than 5 μm from Sigma Aldrich (Cat. No. 310069-500G, St Louis, Mo.). The blowing agents used were sodium bicarbonate (Sigma Aldrich, Cat. No. SX0320-1) and citric acid (VWR, Radnor, Pa., Cat. No. BDH8003-500G). Dimethyl sulfoxide from Fisher Scientific (DMSO, Cat. No. D128-4, Hampton, N.H.) was used without further purification.

Extrusion blending of thermoplastics and magnetite: Different proportions of polymers, magnetite, and blowing agent powders were placed in a glass vessel and mixed in a rotary tumbler (Lortone, Inc. Model 3A) for several minutes. The powder mixtures were fed into a single screw Filabot extruder (Cat. No. EX00334) and further blended this way at different ranges of temperature and speeds depending upon the polymer matrix composition and blowing agent combination. The size of the composite magnetic materials obtained from the extrusion process was reduced in a stainless-steel blender (Waring Commercial Cat. No. CB15T) to a size of 2.82 mm and smaller by grinding them at high speed at −78.5° C. for several minutes various times. Then the material particle size was further reduced to 60 microns and smaller by cooling it at −184.44° C. and feeding the particles to an impact mill (Vortec Model M-1) operating at 20,000 RPM, this process was repeated as many times as required to obtain that particle size.

Extruded composite example 1: A blend of 100 g of polypropylene (46% w/w), magnetite (46% w/w), and sodium bicarbonate (8% w/w) powders were mixed in a glass cylinder for 15 minutes in the rotary tumbler. The solid blended powders were fed to the extruder and mixed at 175° C. and 21 RPM.

Extruded composite example 2: Another blend was made using 100 g of a powder mixture of polystyrene (48% w/w), magnetite (<10 μm, 48% w/w) and sodium bicarbonate (4% w/w). The powders were pre-mixed in the rotary tumbler for 15 min. The mixture was further blended in the extruder at the temperature range of 170° to 175° C. and a speed of 12 RPM.

Thermoplastics and magnetite blends for 3D printing by dissolution coating: powders of nylon 12 and magnetite (<5 μm) 50/50 w/w were added into a glass vessel containing DMSO in a ratio of 90% v/v of solvent to 10% v/v of powder materials. The mixture was homogeneously stirred at 500 RPM and the temperature was gradually raised to 140° C. to allow nylon 12 to dissolve. The material was kept in solution for 15 min. After that time, the mixture of dissolved nylon 12 and dispersed magnetite was allowed to slowly cool down while stirring at 500 RPM. In this fashion, magnetite particles were incorporated into the nylon 12 particles that started to precipitate out of DMSO solution during the cooling down process. The magnetite nylon 12 composite particles were purified using a neodymium magnet to remove as much of the DMSO as possible. Then the composite powder was rinsed with cold water several times, after that the material was dried in a vacuum oven at 100° C. for 24 hours to remove the remaining absorbed DMSO. Finally, the material was sieved using a 100 μm opening mesh.

The microstructural features of the developed composite materials before and after sintering were observed under a field emission scanning electron microscope (FESEM, Tescan Mira3) after being sputter coated (7 nm in thickness) with a gold-palladium target.

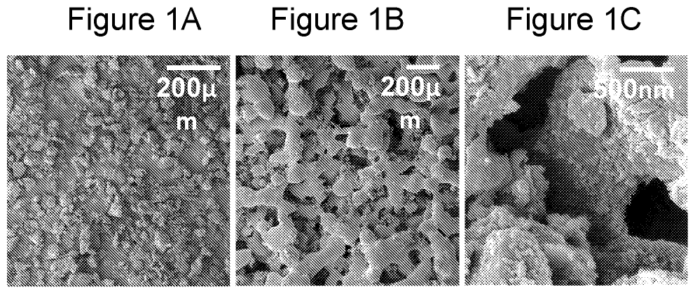

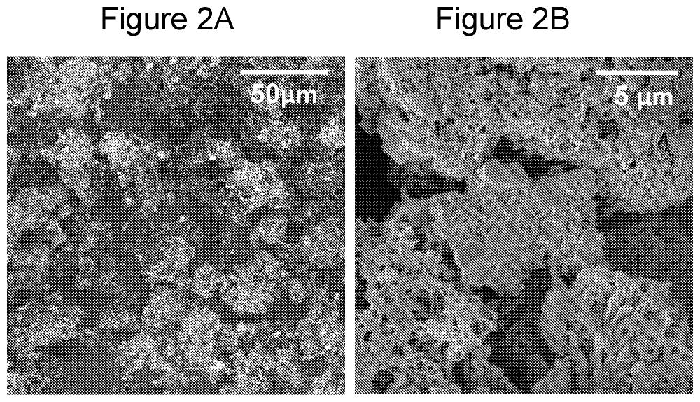

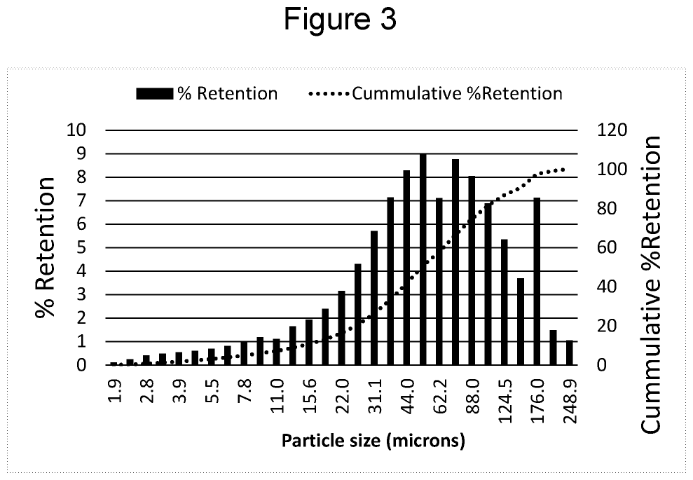

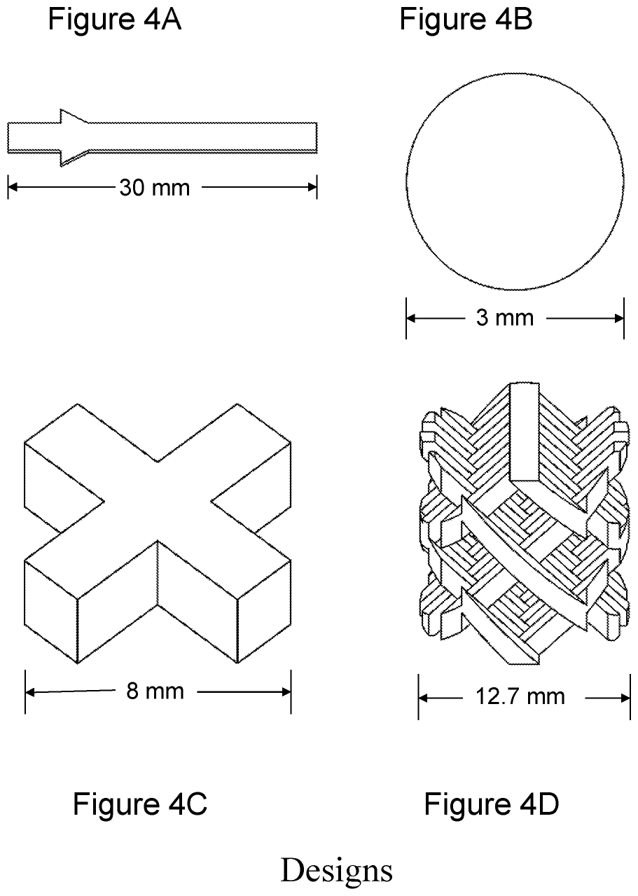

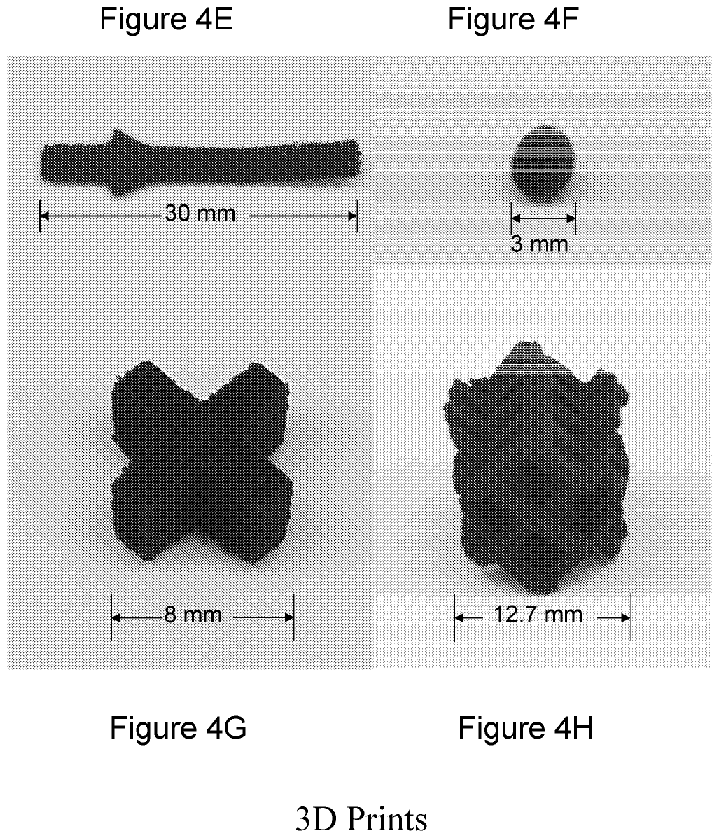

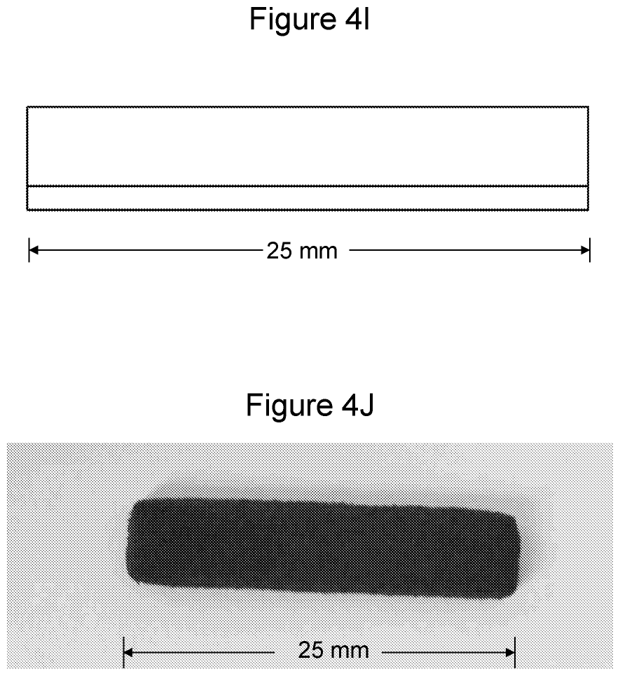

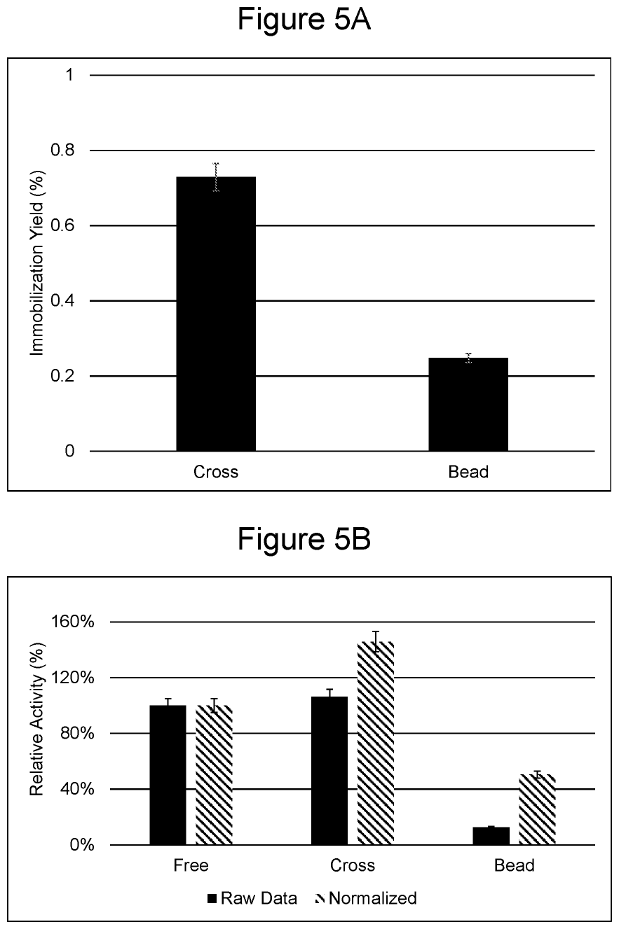

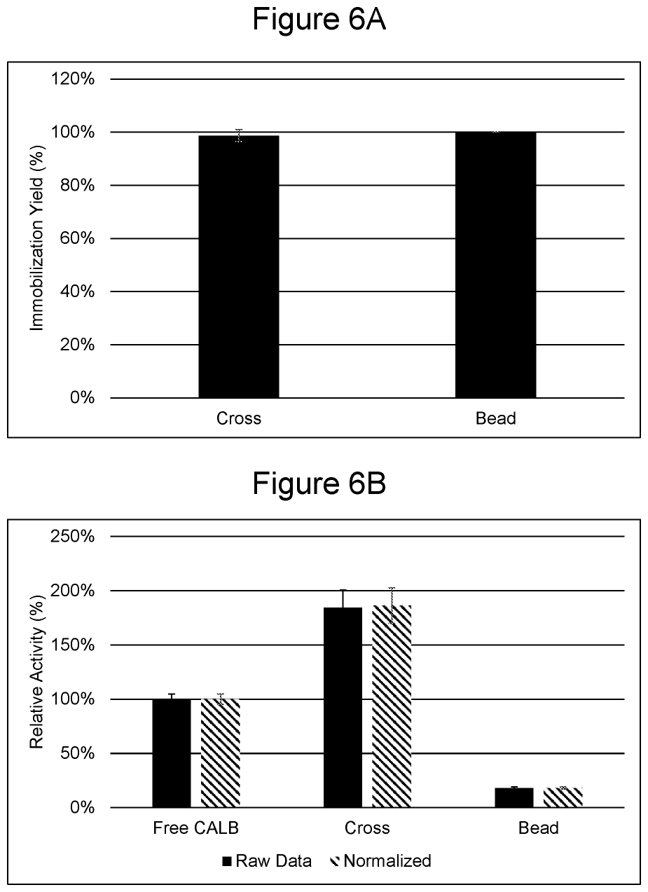

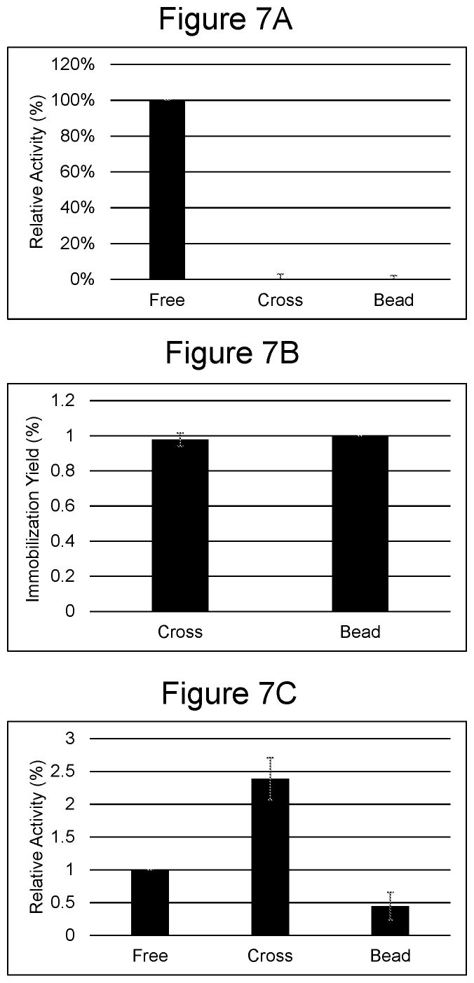

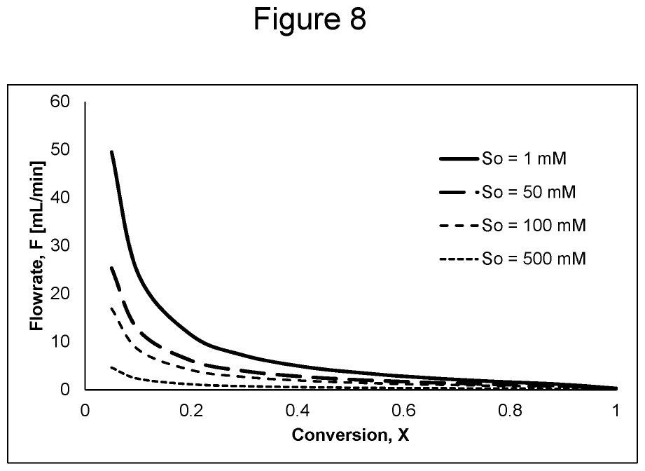

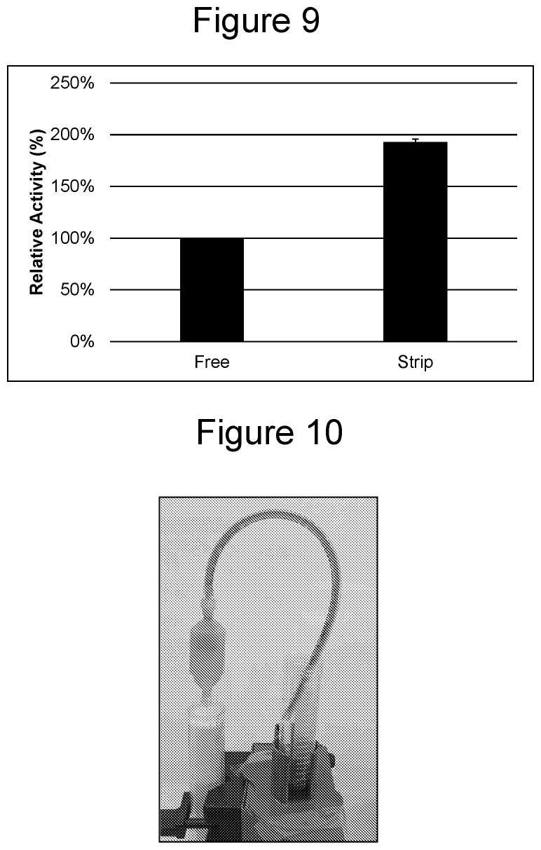

The morphology of the extruded polypropylene magnetite composite after cryogenic grinding, sintering, and enzyme immobilization was evaluated by SEM are shown in FIGS. 1A-1C, respectively. The shape of the cryogenically grinded composite particles is not perfectly round but is rather regular. The particle size distribution of the polypropylene magnetite composite after cryogenic grinding is the range of 1.945 to 248.9 microns (FIG. 3). Particles smaller than 37 microns were below 35% of the total and 60% were below 62.23 microns. For the sintering process, the polypropylene magnetite composite powder was screened with a 100 micron opening mesh. This is consistent with the particles observed in the SEM image (FIG. 1B). The particles below 40 microns were not removed. This maximized the utilization of the cryo-ground composite powders to a 75% of the total. The flowing ability of the powders was good enough for sintering. FIG. 1B displays a successful sintering process of the polypropylene magnetite extruded composite powders that holds the characteristic solidification, coarsening, and porosity features of this process due to the fusing of the powders. Furthermore, it is possible to see residual sodium bicarbonate particles with a needle like shape (approximately 0.5 microns in diameter and 8 microns in length). The sodium bicarbonate residual material was easily removed with a water rinse before enzyme immobilization, also shown by SEM. FIG. 1C (500 nm scale bar) shows the attachment of faceted magnetite nanoparticles (MNP) in a clusters form (level 1) onto the surface of the sintered polypropylene magnetite composite (level 2) after invertase immobilization. This was not previously observed on the sintered material. The density of the MNP is high on the surface of the sintered material. This correlates with the enzymatic immobilization and activity yield results.

FIGS. 2A and 2B show the morphology of the nylon 12 magnetite composite produced from the dissolution coating process. The backscatter SEM image of the material (FIG. 2A) with a 50 micron scale bar shows the magnetite particles with a lighter color. This is due to the higher atomic number of magnetite densely embedded on the surface of nylon 12 powder (dark color particles). The size of the magnetite coated nylon 12 powders was approximately 20 to 25 microns in size when measuring 150 particles from SEM images. The assembly of the composite is further shown in FIG. 2B (5 microns scale bar) as blossom-like or dendritic high surface area nylon 12 particles highly coated with faceted magnetite particles.

### Example 2—Thermoplastic-Magnetite Blend Sintering and 3D Printing

3D printing of the thermoplastic-magnetite composite was conducted with a Sintratec© Kit (https://sintratec.com/, Switzerland). Objects were computer modeled with Autodesk Inventor (https://www.autodesk.com/). Polyamide 12 (Nylon 12, AdSint PA12) was purchased from Advanc3D Materials GmbH. Polypropylene (Polyaxis, Fairlawn, Ohio, Cat. No. PD 2000) sample was obtained from A. Schulman. Iron oxide (Cat. No. QM-D50/4 Magnetite) with a particle size smaller than 10 μm was obtained from Reade Advanced Materials and iron (II, III) oxide particle size smaller than 5 μm from Sigma Aldrich (Cat. No. 310069-500G). The blowing agent used was sodium bicarbonate (EMD Millipore, Cat. NO. SX0320-1). Two types of low-density fumed silica were obtained from Cabot (CAB-O-SIL TS-530, CAB-O-SIL TS-610). An additional type of silica was obtained from Evonik (AEROSIL R 812). A number of properties were optimized, particularly high young's moduli, resolution, void fraction, yield strength, and magnetic moment with low elastic ranges and hydrophobicity. Printing parameters (powder flowability, chamber temperature, print bed temperature and laser speed) were investigated and ensure maximum surface area while maintaining object strength.

Powder Flowability—Flowability testing was conducted using a plate with etched rectangular channels and a flat leveler. Powder was poured onto the plate and the leveler was scraped across the surface of the channel. If the powder was unable to spread evenly, low density fumed silica was incorporated to the composition to improve flowability. Extruded composite blends of polypropylene (46% w/w), magnetite (<10 μm 46% w/w), and sodium bicarbonate (8% w/w) did not require silica for printing but after adding silica (R 812 0.25% w/w) the sample became noticeably more fluid. A physical blend of nylon 12 (49.625% w/w), magnetite (<5 μm 49.625% w/w), BC (0.5% w/w), and silica (TS-610 0.25% w/w) was able to sinter in contrast with its counterpart without added silica. All three silicas were analyzed and similar results were observed. Composites powders with processed base powders and added filler thermoplastics often required additional silica to achieve desired flowability. A range of 0.25-0.75% (w/w) was required to produce spreadable powders. Compositions that did not require the addition of flowing agents, however, were more desirable for the final process.

Chamber Temperature—The chamber temperature refers to the ambient temperature in the printer interior. Before printing, the chamber is heated until the air, powder, and structural components are at a uniform temperature to minimize the effects of heat transfer during printing. Heat transfer between sintered layers and fresh powder can cause the sintered piece to cause print failure. In general, the best chamber temperatures were found within 10° C. of the glass transition temperature. For nylon 12-magnetite composites, the optimal print chamber temperature was 140° C. The chamber temperature for polypropylene-magnetite composites was iterated from 110-130° C. with the optimal chamber temperature being 120° C.

Fabrication Surface Temperature—The fabrication surface temperature is the temperature of the surface of the print bed. The surface temperature is controlled by an infrared temperature sensor and an array of three heat lamps. This parameter is responsible for bringing the surface temperature right below the glass transition temperature. In general, optimal surface temperatures were found within 5° C. of the glass transition temperature. For nylon 12-magnetite composites, optimal print surface temperatures were between 160° and 170° C. depending on the composition. Blends of nylon 12 (49.875% w/w), 49.875% [BC 2% w/w, magnetite <10 μm 98% w/w], and silica (TS-610 0.25% w/w) were found to sinter best with a surface temperature of 164° C. while 79.75% [46% nylon 12, 46% <10 μm magnetite, 8% sodium bicarbonate] (w/w), nylon 12 (20% w/w), and silica (TS-610 0.25% w/w) were best sintered with a surface temperature of 170° C. The surface temperature for polypropylene-magnetite composites was iterated from 110-155° C., with the optimal surface temperature of 130° C.

Laser Scan Speed—This is the speed of laser travel during sintering. This parameter modifies adhesion between layers, pore sizes, material strength, and sintering ability. The laser scan speed is modified to ensure that powders bond together on each layer due to the varying levels to which the powders will absorb the energy from the laser. Laser speed also affects sintering of different samples due to their material composition and packing structure. In some cases, the thermoplastic is not as readily able to interface with itself (i.e., different coatings or concentrations). Thus, it needs a longer exposure time to allow some powder to reach melting temperature and become amorphized to reach sintered counterparts. For this same reason, scan speed affects the pore sizes. The laser speed was varied from 100 to 950 mm/s. Depending on the compositions, laser speeds were adjusted to flash the powders just above the glass transition temperature. For polypropylene and nylon samples, the optimal laser speed was found to be 650 mm/s.

Varying the concentration of polypropylene, magnetite, sodium bicarbonate, and the conditions under which the blends were made led to different optimization parameters. The following three example prints were successfully sintered, developed strong stable prints with rigidity, and the ability to withstand functionalization with BNCs and enzymatic reactions:

a. Extruded composite blend of polypropylene (46% w/w), magnetite (<10 μm 46% w/w), and sodium bicarbonate (8% w/w) at chamber temperature of 110° C., surface temperature of 125° C., laser speed of 650 mm/s
b. Extruded composite blend of polypropylene (48% w/w), magnetite (<10 μm 48% w/w), and sodium bicarbonate (4% w/w) at chamber temperature of 110° C., surface temperature of 125° C., laser speed of 350 mm/s
c. Extruded composite blend of polypropylene (36% w/w), magnetite (<10 μm 56% w/w), and sodium bicarbonate (8% w/w) at chamber temperature of 119° C., surface temperature of 129° C., laser speed of 350 mm/s

The prints vary in microscopic porosity due to not only these print conditions but their material properties. The optimization of print parameters aimed at maximizing functionalization with BNCs and enzymatic reactions.

Varying the concentration of nylon, BC, silica, magnetite, and sodium bicarbonate and the conditions under which the blends were made led to different optimization parameters. The following three example prints were successful in sintering, developing strong stable prints with rigidity, and the ability to withstand functionalization with BNCs and enzymatic reactions:

a. Physical blend of nylon 12 (49.875% w/w), 49.875% [2% BC, 98% magnetite <10 μm] (w/w), and silica (TS-610 0.25% w/w) at chamber temperature of 140° C., surface temperature of 164° C., laser speed of 300 mm/s
b. Physical blend of 99.75% [Dissolution coating of nylon 12 (50% w/w), 50% [BC (2% w/w), magnetite (<10 μm 98% ww)]], silica (TS-610 0.25% w/w) at a chamber temperature of 140° C., surface temperature of 160° C., laser speed of 200 mm/s
c. Physical blend of 79.75% [extruded composite blend of magnetite (<10 μm 46% w/w), nylon 12 (46% w/w), and sodium bicarbonate (8% w/w)], nylon 12 (20% w/w), silica (TS-610 0.25% w/w) at chamber temperature of 140° C., surface temperature of 170° C., laser speed of 300 mm/s.

The prints varied in microscopic porosity due to not only sintering conditions but also material properties such as powder size. The optimization of print parameters aimed at maximizing functionalization with BNCs and enzymatic reactions.

Printing of polypropylene-magnetite objects: Powders of an extruded composite blend of magnetite (<10 μm 46% w/w), polypropylene (46% w/w), and sodium bicarbonate (8% w/w) were prepared as previously described. The powders were placed in the print and reservoir beds of the Sintratec Kit and preheated at 115° C. for one hour. Strips, beads, crosses, and mixing elements were sintered at a 115° C. chamber temperature, 125° C. surface temperature, 650 mm/s laser scan speed, and 100 μm layer height. Once sintering was complete, the printed parts were left in the chamber to slowly cool for one hour. For single layer strips, the length was 30 mm, the width was 3 mm, the thickness was 0.1 mm, and the mass was 0.026 g, the theoretical surface area was 262 mm². For beads, the diameter was 3 mm, the mass was 0.01 g, the volume was 0.014 cm², the theoretical surface area was 0.283 cm². For crosses, the length was 8 mm, the width was 2 mm, the thickness was 5 mm, the mass was 0.11 g, the volume was 0.147 cm³, density of 0.75 grams per cm³, the theoretical surface area was 2.16 cm². For the static mixing element, the length was 12.7 mm, the diameter was 12.7 mm, the mass was 0.35 grams, the volume was 0.46 cm³, and the density was 0.76 grams per cm³. FIGS. 4A-4D show 3D models and FIGS. 4E-4H show high-resolution images of the 3D object samples after printing.

An estimation of the surface area created during the sintering process of the polypropylene magnetite composite was done using the following rationale. The values are measured by mercury porosimetry and BET Brunauer-Emmett-Teller (BET) surface characterization. The theoretical density of the composite was calculated using equation 1 (Sharma, J. Materials Engin. and Performance 12:324-330 (2003)):

ρ_{composite}=m_{C}/v_{C}=(m_{P}+m_{M})/(v_{P}+v_{M}) (eq. 1)

Where m_{C} is the composite mass, v_{C} the composite volume, m_{P} is the mass reinforcing particles, m_{M} the mass of the polymer matrix, v_{P} the volume of reinforcing particles and v_{M} the polymer matrix volume. Using the density of polypropylene as 0.92 g/cm₃ and 5 g/cm₃ for magnetite in a 50 to 50 polypropylene to magnetite weight ratio, the respective volumes of polypropylene and magnetite relative to 2 grams of material are 1.087 cm³ and 0.2 cm³. Therefore, the density of the composite is 1.55 g/cm³. The theoretical porosity percentage of the 3D printed sintered materials can be calculated by using the previously calculated composite density (ρcomposite) and the density of the actual sintered materials (ρsm) as follow (equation 2):

% Porosity=100×[1−(ρₛₘ/ρ_{composite})] (eq. 2)

The sintered material density was determined as 0.75 g/cm³ by dividing the mass of the sintered object by the actual solid volume of the design (calculated by the CAD software). Hence the theoretical porosity of the sintered materials was 52%. This way, the volume occupied by the actual composite particles was estimated by multiplying the solid fraction (0.48) by the volume of the sintered material. Then, assuming a spherical shape of PP-Mag particles and an average diameter of 34 microns, calculate the particle size and its respective percentage in the utilized size range. The amount of particles used to create that volume was calculated by dividing the solid volume of the sintered object by the volume of a single particle. Then the obtained particles were reduced to two thirds of the total, assuming one third of them lost their shape by solidification during the sintering process. Multiplying the surface area of one single particle (SA=3V/r, 3.64×10⁻⁴ cm²) by the total amount of available particles in the sintered 3D object provided the theoretical expected surface area. This was expected to be higher since the surface area created by the porosity and surface roughness was not considered in this approximation. The average specific surface area was calculated as 762 cm² per gram of sintered material.

It was demonstrated that 3D magnetic objects were able to be printed (FIGS. 4E-4H) with the powder composition of magnetite blended with thermoplastics so that they maintained rigidity and porosity for the immobilization of enzymes. Objects were used to immobilize BNCs.

The printed geometries (FIGS. 4A-4D) were designed for different applications. Strips were designed and engineered for quick optimization of the immobilization of enzymes. Beads were designed and engineered for the immobilization of BNCs and to fit comfortably in 96-well microplates for the screening of enzyme activities. Their shape allows for easy removal from the wells with external magnets. Crosses were designed and engineered for the immobilization of BNCs in small and medium batch reactors. Crosses can also be used as stir elements when combined with stir plates. Static mixers were designed and engineered for immobilization of BNCs in continuous flow systems. Static mixers consist of individual mixing elements placed in series. Adjacent elements are rotated 90° with respect to each other. Pipe housing encloses the static mixer and provides connections within continuous flow systems.

### Example 3—Immobilization of Bionanocatalysts (BNCs) on 3D Printed Scaffolds

Invertase Immobilization on Sintered Materials: Invertase from baker's yeast was purchased from Sigma Aldrich (St. Louis, Mo., USA, Cat. No. 14504). All water was obtained from a BarnStead Nanopure water purifier (Thermo Scientific, 18.2 MOhm-cm). Iron oxide nanoparticles (NPs) were prepared as previously described (U.S. Pat. No. 9,597,672; Int'l Pub. No. WO2018102319, each of which are incorporated reference herein in their entirety) and stored under a N₂ sparged atmosphere at pH 11 at 4° C.

Nanoparticles were ultrasonicated (Fisher Scientific) on the day of immobilization for 1 minute at 40% amplitude. NP solutions of 1000 μg/mL were prepared from a 24 mg/mL stock solution with pH 3 water. Cold NP solution was added rapidly to cold enzyme solution containing: 200 μg/mL of invertase in pH 7 water. The NP-enzyme mix was then added to a centrifuge tube (Eppendorf) and incubated for 1 hour at 25° C. The immobilization yield was quantified by the Bradford method (Bradford reagent: Quick Start™ Bradford 1× Dye Reagent #5000205 from BioRad) by comparing against invertase enzyme standards.

A reducing sugar substrate assay was developed to assess the activity of free and immobilized invertase. The assay measured the hydrolysis of raffinose into fructose and melibiose using 4-hydroxybenzhydrazide (PAHBAH) purchased from Sigma Aldrich (cat. H9882). D-(+)-Raffinose pentahydrate (Cat No. R0250) was purchased from Fisher Scientific (Hampton, N.H., USA, Cat. No. AAA1831309) and D-(+)-glucose was purchased from EMD Millipore (Cat. No. DX0145-3). 10×PBS buffer was purchased from Thermo Scientific (Waltham, Mass., USA, Cat. SH30258.02).

A reaction mix was prepared with 1 mM raffinose in 1×PBS buffer (pH 7.4) and pH 7 water. Reaction mix was added to the immobilized and free enzyme systems, then incubated for 5 minutes at 25° C. The resulting supernatant was collected and added to a 5 mg/mL solution of PAHBAH in 0.5 M NaOH (cat. 7708-10) purchased from Macron Fine Chemicals (Center Valley, Pa., USA). The supernatant-PAHBAH mix was then boiled for 5 minutes and quantified via absorbance at 410 nm and compared to a standard curve of glucose.

The immobilization yields of invertase on the 3D printed cross and bead were 75% and 25%, respectively (FIG. 5A). The relative activities of immobilized invertase for the hydrolysis of raffinose by the cross and bead were 146% and 50%, respectively, when normalized by the immobilization yield (FIG. 5B).

It was demonstrated that invertase immobilized on the 3D printed cross had comparable activity to free invertase. When normalized, the invertase immobilized on the cross demonstrated a 1.5-fold increase in activity for the hydrolysis of raffinose compared to free invertase.

CALB Immobilization on Sintered Materials: Lipase B from Candida antarctica (CALB) was purchased from Sigma Aldrich (Cat. No. 62288). All water was obtained from a BarnStead Nanopure water purifier (Thermo Scientific, 18.2 MOhm-cm). Iron oxide nanoparticles (NPs) were prepared as previously described (U.S. Pat. No. 9,597,672; Intl Pub. No. WO2018102319, each of which are incorporated by reference herein in their entirety) and stored under a N₂ sparged atmosphere at pH 11 at 4° C.

Nanoparticles were ultrasonicated (Fisher Scientific) on the day of immobilization for 1 min at 40% amplitude. NP solutions of 500 μg/mL were prepared from a 24 mg/mL stock solution with pH 3 water. Cold NP solution was added rapidly to cold enzyme solution containing: 200 μg/mL of CALB in pH 7 water. The NP-enzyme mix was then added to a centrifuge tube (Eppendorf) and incubated for 2 hours at 25° C. The immobilization yield was quantified by the Bradford method (Bradford reagent: Quick Start™ Bradford 1× Dye Reagent #5000205 from BioRad) by comparing against CALB enzyme standards.

To assess the activity of immobilized and free CALB, a colorimetric assay of glycerol ester hydrolysis was developed. The assay measured the hydrolysis of para-nitrophenyl laurate (p-NPL) into para-nitrophenol (p-NP) and lauric acid. The p-NPL was purchased from Fisher Scientific (cat. 50-014-39022) and p-NP was purchased from Sigma Aldrich (cat. 48549).

A reaction mixture was prepared with 100 μM p-NPL in 50 mM Tris (pH 7.5) (cat. RGF-3350) purchased from KD Medical (Columbia, Md., USA). The reaction mixture was added to the immobilized and free enzyme systems, then incubated for 4 minutes at 25° C. The supernatant was collected, then p-NP was quantified by measuring absorbance at 400 nm and compared to a standard of p-NP.

The immobilization yields of CALB on the 3D printed cross and bead (in triplicate) were 99%±2% and 100%, respectively (FIG. 6A). The relative activities of immobilized CALB for the hydrolysis of p-NPL by the cross and bead were 186%±16% and 18%±1%, respectively, when normalized by the immobilization yield. (FIG. 6B).

It was demonstrated that CALB immobilized on the 3D printed cross had comparable activity to free CALB. When normalized, the CALB immobilized on the cross demonstrated a 1.8-fold increase in activity for the hydrolysis of p-NPL compared to free CALB.

Reuse of 3D Scaffold Sintered Materials with CALB: The 3D printed crosses and beads that were previously immobilized with 100 μg/mL CALB and 250 μg/mL NPs were boiled in pH 2 water obtained from a BarnStead Nanopure water purifier (Thermo Scientific, 18.2 MOhm-cm) for 20 minutes to remove any enzymes/NPs. To validate that all enzymes and NPs were removed from the crosses and beads, the previously described colorimetric assay of glycerol ester hydrolysis was used. The assay measured the hydrolysis of para-nitrophenyl laurate (p-NPL) into para-nitrophenol (p-NP) and lauric acid. The p-NPL was purchased from Fisher Scientific (cat. 50-014-39022) and p-NP was purchased from Sigma Aldrich (cat. 48549). A reaction mix was prepared with 100 μM p-NPL in 50 mM Tris (pH 7.5) (cat. RGF-3350) purchased from KD Medical (Columbia, Md., USA). The reaction mix was added to the immobilized and free enzyme systems then incubated for 4 minutes at 25° C. The supernatant was collected then p-NP was quantified by measuring absorbance at 400 nm and compared to a standard of p-NP.

The washed crosses and beads were used to re-immobilize Lipase B from Candida antarctica (CALB) purchased from Sigma Aldrich (cat. 62288). All water was obtained from a BarnStead Nanopure water purifier (Thermo Scientific, 18.2 MOhm-cm). Iron oxide nanoparticles (NPs) were prepared as previously described (U.S. Pat. No. 9,597,672; Intl Pub. No. WO2018102319, each of which are incorporated by reference herein in their entirety) and stored under a N₂ sparged atmosphere at pH 11 at 4° C.

Nanoparticles were ultrasonicated (Fisher Scientific) on the day of immobilization for 1 minute at 40% amplitude. NP solutions of 500 μg/mL were prepared from a 24 mg/mL stock solution with pH 3 water. Cold NP solution was added rapidly to cold enzyme solution containing: 200 μg/mL of CALB in pH 7 water. The NP-enzyme mix was then added to a centrifuge tube (Eppendorf) and incubated for 2 hours at 25° C. The immobilization yield was quantified by the Bradford method (Bradford reagent: Quick Start™ Bradford 1× Dye Reagent #5000205 from BioRad) by comparing against CALB enzyme standards.

To assess the activity of immobilized and free CALB, a colorimetric assay of glycerol ester hydrolysis was developed. The assay measured the hydrolysis of para-nitrophenyl laurate (p-NPL) into para-nitrophenol (p-NP) and lauric acid. The p-NPL was purchased from Fisher Scientific (cat. 50-014-39022) and p-NP was purchased from Sigma Aldrich (cat. 48549). A reaction mix was prepared with 100 μM p-NPL in 50 mM Tris (pH 7.5) (cat. RGF-3350) purchased from KD Medical (Columbia, Md., USA). The reaction mix was added to the immobilized and free enzyme systems, then incubated for 4 minutes at 25° C. The supernatant was collected, then p-NP was quantified by measuring absorbance at 400 nm and compared to a standard of p-NP.

The relative activity of CALB on the 3D printed cross and bead after washing (in triplicate) were 0%±3% and 0%±2%, respectively (FIG. 7A). The immobilization yields of re-immobilized CALB on the cross and bead were 98%±4% and 100%, respectively (FIG. 7B). The activity of the fresh immobilized CALB was about 2-fold higher than the free enzyme (FIG. 7C). The results of the activity assay on the washed crosses and beads indicate that 3D printed scaffolds can be cleared of previously immobilized enzymes and NPs. Furthermore, the scaffolds can be re-functionalized with fresh enzymes with minimal losses to the immobilization yields.

### Example 4—BM3 P450 Immobilization on Sintered Materials

Recombinant BM3 Cytochrome p450, recombinant glucose dehydrogenase (GDH), Bovine serum albumin (BSA), Bovine liver catalase (CAT), Bovine erythrocyte cytosolic superoxide dismutase (SOD) expressed in E. coli, glucose (beta-d-glucose), p-nitrophenyl laurate (p-NPL), p-nitrophenol (p-NP), nicotinamide adenine dinucleotide phosphate (reduced) tetrasodium salt (NADPH), are immobilized on the 3D printed scaffolds. All stock solutions are made with 18.2 Me-cm water purified by Barnstead™ Nanopure™ water purifier (Thermo Scientific). Absorbance is measured in triplicate in Costar™ 3635 UV-transparent microplates using Biotek Synergy4™ plate reader operating with Gen5™ software. A sonicator (FB-505) with a″ inch probe from Fisher Scientific® (Waltham, Mass.) is used for all sonication. Iron oxide nanoparticles (NPs) were prepared as previously described (U.S. Pat. No. 9,597,672; Intl Pub. No. WO2018102319, each of which are incorporated by reference herein in their entirety) and Universal enzyme immobilization within hierarchically-assembled magnetic scaffolds. Catalysis & Biocatalysis 2016; 34 each of which are incorporated by reference herein in their entirety) and stored under a N₂ sparged atmosphere at pH 11 at 4° C.

All aqueous stocks are prepared with ultrapure (MQ) water. Lyophilized BM3-P450, GDH, and NADPH are dissolved in ice-cold oxygen free 2 mM PBS, pH 7.4 and prepared fresh daily. BM3-P450 and GDH are centrifuged at 4° C. at 12000 g for 10 min to pellet cell debris. Their supernatants are collected, and protein content is quantified using the Bradford assay (Bradford reagent: Quick Start™ Bradford 1× Dye Reagent #5000205 from BioRad) with BSA standards. p-NPL and p-NP stock solutions are prepared in pure DMSO to 100 mM and stored at 4° C. Magnesium chloride (1M) and glucose (100 mM) are dissolved in water and stored at 4° C. All stock solutions are kept on ice. Dilutions are made just before use in assays and allowed to equilibrate to room temperature (21° C.).

BM3-P450 immobilizations are optimized using the method described U.S. Pat. No. 9,597,672 and Intl Pub. No. WO2018102319, each of which are incorporated by reference herein in their entirely. The non-BM3-P450 biological and chemical component to the immobilization are referred to as the P450 Support System (SS): GDH for cofactor regeneration, CAT/SOD for reactive oxygen species (ROS) control, and NADPH for stability during immobilization. Free BM3-P450/GDH/CAT/SOD/NADPH stock (500 μg/mL BM3-P450, 100:100:1:1:100 molar ratios) is prepared in cold buffer using fresh enzyme stocks. A 2500 μg/ml NP stock is sonicated at the 40% amplitude for 1 min, equilibrated to room temperature using a water bath, and its pH is adjusted to 3. Free BM3-P450+SS is dispensed into a microcentrifuge tube to which an equal volume of sonicated NPs is added, then pipette mixed 10 times. The BM3-P450+SS system is immobilized by adding BM3-P450+SS to 3D printed scaffolds (crosses and beads) and incubated for 1 hour before it is pelleted magnetically. The immobilization yield is quantified by the Bradford and compared against corresponding enzyme standards.

BM3-P450 activity determination methods are based on BM3-P450-catalyzed oxidation of p-NPL to form p-NP and ω-1 hydroxylauric acid. Enzyme activity is measured by the increase in absorbance at 410 nm due to the formation of p-NP. BM3-P450 reactions are run at 2° C. for 18 hours in microcentrifuge tubes containing 100 mM pH 8.2 phosphate buffered saline (PBS), 0.25 mM p-NPL (0.25% DMSO), 0.15 mM NADPH, 1 mM magnesium chloride, 1 mM glucose, and 3.6 μg/mL CYP (˜60 nM). Free enzyme controls also contain 60 nM GDH. The supernatant is collected, then p-NP is quantified by measuring absorbance at 400 nm and compared to a standard of p-NP. One unit (U) of CYP-394 activity is defined as 1 μmol p-NP generated per minute at 21° C. in 100 mM PBS (pH 8.2).

The BM3-P450+SS system is fully immobilized onto 3D printed scaffolds (crosses and beads). The activity is compared to the free enzyme system to show that the 3D printed scaffolds are efficacious in immobilizing and retaining enzymatic activity with multi-enzyme systems and systems requiring cofactor recycling.

### Example 5—Invertase Immobilization on 3D Printed Flow Cells

3D printed scaffolds designed for flow cells immobilize invertase. All water is obtained from a BarnStead Nanopure water purifier (Thermo Scientific, 18.2 MOhm-cm). Iron oxide nanoparticles (NPs) are prepared as previously described (U.S. Pat. No. 9,597,672; Int'l Pub, No, WO2018102319, each of which are incorporated by reference herein in their entirety) and stored under a N₂ sparged atmosphere at pH 11 at 4° C.

Nanoparticles are ultrasonicated (Fisher Scientific) on the day of immobilization for 1 min at 40% amplitude. NP solutions of 500 μg/mL are prepared from a 24 mg/mL stock solution with pH 3 water. For a 1-gram flow cell, 52 mL of cold NP solution is added rapidly to 52 mL of a cold enzyme solution containing 200 μg/mL of invertase in pH 7 water. The NP-enzyme mix is then added to the 3D printed flow cell and incubated for 2 hours at 25° C. The immobilization yield is quantified by the Bradford method by comparing it to invertase enzyme standards.

The activity of the flow cell is determined by the previously described method of sucrose (So) hydrolysis. The flow cell acts as a packed bed reactor (PBR) and Michaelis-Menten kinetics parameters of Kₘ=49 mM and Vₘₐₓ=127 mM/min (Combes et al., Carbohydrate Res. 117(6):215-228 (1983) incorporated by reference herein in its entirety). The flow rate (F) of reaction mix containing 1 M sucrose in 1×PBS buffer (pH 7.5) is 0.088 mL/min.

As determined by the Bradford method, the invertase is completely immobilized onto the flow cell with 100% activity being retained. The conversion corresponds to the fundamental PBR mass balance based on a Michaelis-Menten kinetic model to determine the solution space for the flow and loading condition for optimal conversion (FIG. 8):

Vₘₐₓ/F=[S]oX−Kₘ*ln(1−X)

Given the previous parameters, the conversion (X) of sucrose to glucose and fructose is 99.99%. The residence time is based on the volume in the flow cell, so for a 100 mL reaction mix in a 10 mL flow cell, the residence time is 114 minutes.

The foregoing would indicate that 3D printed scaffolds used as flow cells are efficacious both in terms of immobilization yields and retained activity.

### Example 6—HRP Immobilization on Sintered Materials

HRP immobilized on sintered materials retains its activity. Horseradish peroxidase (HRP) (cat. 77332) was purchased from Sigma Aldrich (St. Louis, Mo., USA). All water was obtained from a BarnStead Nanopure water purifier (Thermo Scientific, 18.2 MOhm-cm). Iron oxide nanoparticles (NPs) were prepared as previously described (Corgie S C, Brooks R T, Chairil R, Chun M S, Xie B, Giannelis E P. Universal enzyme immobilisation within hierarchically-assembled magnetic scaffolds. Catalysis & Biocatalysis 2016; 34) and stored under a N₂ sparged atmosphere at pH 11 at 4° C.

Nanoparticles were ultrasonicated (Fisher Scientific) on the day of immobilization for 1 min at 40% amplitude. Nanoparticle solutions of 5000 μg/mL were prepared from a 24 mg/mL stock solution with pH 9 water. Cold NP solution was added rapidly to a cold enzyme solution containing: 1000 μg/mL of HRP in pH 7 water. The NP-enzyme mix was then added to a centrifuge tube (Eppendorf) along with a 3D-printed strip and incubated for 2 hrs at 25° C. The immobilization yield was quantified by the Bradford method (Bradford reagent: Quick Start™ Bradford 1× Dye Reagent #5000205 from BioRad) by comparing against HRP enzyme standards.

To assess the activity of immobilized and free HRP, a colorimetric assay of hydrogen peroxide was developed. The assay measured the ω-oxidation of 4-aminoantipyrine (4-AAP) and phenol by hydrogen peroxide. The phenol (cat. W322318) and 4-AAP (cat. A4382) was purchased from Sigma Aldrich (St. Louis, Mo., USA). A reaction mix was prepared with 1 mM hydrogen peroxide, 0.5 mM 4-AAP, and 0.5 mM phenol in 500 mM PBS (pH 7.4) (cat. SH30258.02) was purchased from Thermo Scientific (Waltham, Mass., USA).

The reaction mix was added to the immobilized and free enzyme systems, then incubated for 3 minutes at 25° C. The supernatant was collected, then the product (pink coloured quinoneimine dye) was quantified by measuring the absorbance at 500 nm.

The immobilization yields of HRP on the 3D printed strip (in triplicate) were 95%±1%. The relative activity of immobilized HRP to free enzyme for the ω-oxidation of 4-AAP and phenol was 193%±3%, when normalized by the immobilization yield. (FIG. 9).

### Example 7—HRP Immobilization on 3D Printed Flow Cells

Immobilized HRP retains its activity in a 3D printed flow cell as determined by a colorimetric assay. Horseradish peroxidase (HRP) (cat. 77332) was purchased from Sigma Aldrich (St. Louis, Mo., USA). All water was obtained from a BarnStead Nanopure water purifier (Thermo Scientific, 18.2 MOhm-cm). Iron oxide nanoparticles (NPs) were prepared as previously described (Corgie S C, Brooks R T, Chairil R, Chun M S, Xie B, Giannelis E P. Universal enzyme immobilisation within hierarchically-assembled magnetic scaffolds. Catalysis & Biocatalysis 2016; 34) and stored under a N2 sparged atmosphere at pH 11 at 4° C.

Nanoparticles were ultrasonicated (Fisher Scientific) on the day of immobilization for 1 min at 40% amplitude. Nanoparticle solutions of 5000 μg/mL were prepared from a 24 mg/mL stock solution with pH 9 water. Cold NP solution was added rapidly to a cold enzyme solution containing: 1000 μg/mL of HRP in pH 7 water. The NP-enzyme mix was then added to a centrifuge tube (Eppendorf) along with a 3D-printed strip and incubated for 2 hrs at 25° C. The immobilization yield was quantified by the Bradford method (Bradford reagent: Quick Start™ Bradford 1× Dye Reagent #5000205 from BioRad) by comparing against HRP enzyme standards.

After mixing, the resulting solution was circulated through the flow cell for 1 hour. To assess the activity of immobilized and free HRP, a colorimetric assay of hydrogen peroxide was developed. The assay measured the ω-oxidation of 4-aminoantipyrine (4-AAP) and phenol by hydrogen peroxide. The phenol (cat. W322318) and 4-AAP (cat. A4382) was purchased from Sigma Aldrich (St. Louis, Mo., USA).

A reaction mix was prepared with 1 mM hydrogen peroxide, 0.5 mM 4-AAP, and 0.5 mM phenol in 500 mM PBS (pH 7.4) (cat. SH30258.02) was purchased from Thermo Scientific (Waltham, Mass., USA). The reaction mix was added to the immobilized and free enzyme systems, then incubated for 3 minutes at 25° C. The supernatant was collected, then the product (pink colored quinoneimine dye) was quantified by measuring the absorbance at 500 nm. Identical reaction mix was pumped through the flow cell via syringe pump (NE-100, New Era Pump Systems Inc., Farmingdale, N.Y.) at 2 mL/min to colorimetrically determine the activity of the immobilized HRP (FIG. 10). HRP was successfully immobilized on a 3D printed flow cell and retained activity as indicated by the colorimetric assay.

### Example 8—Epimerase Immobilization on 3D Printed Flow Cells

Epimerase was immobilized onto 3D printed scaffolds designed as flow cells. All water was obtained from a BarnStead Nanopure water purifier (Thermo Scientific, 18.2 MOhm-cm). Iron oxide nanoparticles (NPs) were prepared as previously described in Corgie, et al., Catalysis & Biocatalysis 34(5):15-20 (2016), incorporated by reference herein in its entirety. They were stored under a N₂ sparged atmosphere at pH 11 at 4° C.

Nanoparticles were ultrasonicated (Fisher Scientific) on the day of immobilization for 1 min at 40% amplitude. NP solutions of 8.8 mg/mL were prepared from an 8.9 mg/mL stock solution with pH 3 water. For a 1.8 gram flow cell, 26.2 mL of cold NP solution was added rapidly to 26.2 mL of a cold enzyme solution containing: 3.5 mg/mL of epimerase in pH 8 water with 4 mM Tris (pH 7.5) (cat. RGF-3350) purchased from KD Medical (Columbia, Md., USA). The NP-enzyme mix was then circulated through a 6 mL 3D printed flow cell at 5 mL/min and incubated for 2 hr at 25° C. The immobilization yield was quantified by the Bradford method (Bradford reagent: Quick Start™ Bradford 1× Dye Reagent #5000205 from BioRad) by comparing it against epimerase enzyme standards. The Bradford analysis showed that 87%±1% of the epimerase was immobilized onto the flow cell.

Exemplary Sequences
Bifunctional P450/NADPH-P450 reductase [Bacillus
megaterium]
SEQ ID NO: 1
MTIKEMPQPKTFGELKNLPLLNTDKPVQALMKIADELGEIFKFEAPGRVTRYLSSQRL
IKEACDESRFDKNLSQALKFVRDFAGDGLFTSWTHEKNWKKAHNILLPSFSQQAMK
GYHAMMVDIAVQLVQKWERLNADEHIEVPEDMTRLTLDTIGLCGFNYRFNSFYRDQ
PHPFITSMVRALDEAMNKLQRANPDDPAYDENKRQFQEDIKVMNDLVDKIIADRKA
SGEQSDDLLTHMLNGKDPETGEPLDDENIRYQIITFLIAGHETTSGLLSFALYFLVKNP
HVLQKAAEEAARVLVDPVPSYKQVKQLKYVGMVLNEALRLWPTAPAFSLYAKEDT
VLGGEYPLEKGDELMVLIPQLHRDKTIWGDDVEEFRPERFENPSAIPQHAFKPFGNG
QRACIGQQFALHEATLVLGMMLKHFDFEDHTNYELDIKETLTLKPEGFVVKAKSKKI
PLGGIPSPSTEQSAKKVRKKAENAHNTPLLVLYGSNMGTAEGTARDLADIAMSKGF
APQVATLDSHAGNLPREGAVLIVTASYNGHPPDNAKQFVDWLDQASADEVKGVRY
SVFGCGDKNWATTYQKVPAFIDETLAAKGAENIADRGEADASDDFEGTYEEWREH
MWSDVAAYFNLDIENSEDNKSTLSLQFVDSAADMPLAKMHGAFSTNVVASKELQQ
PGSARSTRHLEIELPKEASYQEGDHLGVIPRNYEGIVNRVTARFGLDASQQIRLEAEEE
KLAHLPLAKTVSVEELLQYVELQDPVTRTQLRAMAAKTVCPPHKVELEALLEKQAY
KEQVLAKRLTMLELLEKYPACEMKFSEFIALLPSIRPRYYSISSSPRVDEKQASITVSV
VSGEAWSGYGEYKGIASNYLAELQEGDTITCFISTPQSEFTLPKDPETPLIMVGPGTGV
APFRGFVQARKQLKEQGQSLGEAHLYFGCRSPHEDYLYQEELENAQSEGIITLHTAFS
RMPNQPKTYVQHVMEQDGKKLIELLDQGAHFYICGDGSQMAPAVEATLMKSYADV
HQVSEADARLWLQQLEEKGRYAKDVWAG
Cytochrome P450 3A4 isoform 1 [Homo sapiens]
SEQ ID NO: 2
MALIPDLAMETWLLLAVSLVLLYLYGTHSHGLFKKLGIPGPTPLPFLGNILSYHKGFC
MFDMECHKKYGKVWGFYDGQQPVLAITDPDMIKTVLVKECYSVFTNRRPFGPVGF
MKSAISIAEDEEWKRLRSLLSPTFTSGKLKEMVPIIAQYGDVLVRNLRREAETGKPVT
LKDVFGAYSMDVITSTSFGVNIDSLNNPQDPFVENTKKLLRFDFLDPFFLSITVFPFLIP
ILEVLNICVFPREVTNFLRKSVKRMKESRLEDTQKHRVDFLQLMIDSQNSKETESHKA
LSDLELVAQSIIFIFAGYETTSSVLSFIMYELATHPDVQQKLQEEIDAVLPNKAPPTYD
TVLQMEYLDMVVNETLRLFPIAMRLERVCKKDVEINGMFIPKGVVVMIPSYALHRD
PKYWTEPEKFLPERFSKKNKDNIDPYIYTPFGSGPRNCIGMRFALMNMKLALIRVLQ
NFSFKPCKETQIPLKLSLGGLLQPEKPVVLKVESRDGTVSGA
Cytochrome P450 1A2 [Homo sapiens]
SEQ ID NO: 3
MALSQSVPFSATELLLASAIFCLVFWVLKGLRPRVPKGLKSPPEPWGWPLLGHVLTL
GKNPHLALSRMSQRYGDVLQIRIGSTPVLVLSRLDTIRQALVRQGDDFKGRPDLYTS
TLITDGQSLTFSTDSGPVWAARRRLAQNALNTFSIASDPASSSSCYLEEHVSKEAKALI
SRLQELMAGPGHFDPYNQVVVSVANVIGAMCFGQHFPESSDEMLSLVKNTHEFVET
ASSGNPLDFFPILRYLPNPALQRFKAFNQRFLWFLQKTVQEHYQDFDKNSVRDITGA
LFKHSKKGPRASGNLIPQEKIVNLVNDIFGAGFDTVTTAISWSLMYLVTKPEIQRKIQ
KELDTVIGRERRPRLSDRPQLPYLEAFILETFRHSSFLPFTIPHSTTRDTTLNGFYIPKKC
CVFVNQWQVNHDPELWEDPSEFRPERFLTADGTAINKPLSEKMMLFGMGKRRCIGE
VLAKWEIFLFLAILLQQLEFSVPPGVKVDLTPIYGLTMKHARCEHVQARLRFSIN
CYP2D6 [Homo sapiens]
SEQ ID NO: 4
MGLEALVPLAMIVAIFLLLVDLMHRRQRWAARYPPGPLPLPGLGNLLHVDFQNTPY
CFDQLRRRFGDVFSLQLAWTPVVVLNGLAAVREALVTHGEDTADRPPVPITQILGFG
PRSQGRPFRPNGLLDKAVSNVIASLTCGRRFEYDDPRFLRLLDLAQEGLKEESGFLRE
VLNAVPVLLHIPALAGKVLRFQKAFLTQLDELLTEHRMTWDPAQPPRDLTEAFLAE
MEKAKGNPESSFNDENLCIVVADLFSAGMVTTSTTLAWGLLLMILHPDVQRRVQQEI
DDVIGQVRRPEMGDQAHMPYTTAVIHEVQRFGDIVPLGVTHMTSRDIEVQGFRIPKG
TTLITNLSSVLKDEAVWEKPFRFHPEHFLDAQGHFVKPEAFLPFSAGRRACLGEPLAR
MELFLFFTSLLQHFSFSVPTGQPRPSHHGVFAFLVTPSPYELCAVPR
Cytochrome P450-2E1 [Homo sapiens]
SEQ ID NO: 5
MSALGVTVALLVWAAFLLLVSMWRQVHSSWNLPPGPFPLPIIGNLFQLELKNIPKSF
TRLAQRFGPVFTLYVGSQRMVVMHGYKAVKEALLDYKDEFSGRGDLPAFHAHRDR
GIIFNNGPTWKDIRRFSLTTLRNYGMGKQGNESRIQREAHFLLEALRKTQGQPFDPTF
LIGCAPCNVIADILFRKHFDYNDEKFLRLMYLFNENFHLLSTPWLQLYNNFPSFLHYL
PGSHRKAIKNVAEVKEYVSERVKEHHQSLDPNCPRDLTDCLLVEMEKEKHSAERLY
TMDGITVTVADLFFAGTETTSTTLRYGLLILMKYPEIEEKLHEEIDRVIGPSRIPAIKDR
QEMPYMDAVVHEIQRFITLVPSNLPHEATRDTIFRGYLIPKGTVVVPTLDSVLYDNQE
FPDPEKFKPEHFLNENGKFKYSDYFKPFSTGKRVCAGEGLARMELFLLLCAILQHFNL
KPLVDPKDIDLSPIHIGFGCIPPRYKLCVIPRS
Cytochrome P450-2E1 [Homo sapiens]
SEQ ID NO: 6
MSALGVTVALLVWAAFLLLVSMWRQVHSSWNLPPGPFPLPIIGNLFQLELKNIPKSF
TRLAQRFGPVFTLYVGSQRMVVMHGYKAVKEALLDYKDEFSGRGDLPAFHAHRDR
GIIFNNGPTWKDIRRFSLTTLRNYGMGKQGNESRIQREAHFLLEALRKTQGQPFDPTF
LIGCAPCNVIADILFRKHFDYNDEKFLRLMYLFNENFHLLSTPWLQLYNNFPSFLHYL
PGSHRKAIKNVAEVKEYVSERVKEHHQSLDPNCPRDLTDCLLVEMEKEKHSAERLY
TMDGITVTVADLFFAGTETTSTTLRYGLLILMKYPEIEEKLHEEIDRVIGPSRIPAIKDR
QEMPYMDAVVHEIQRFITLVPSNLPHEATRDTIFRGYLIPKGTVVVPTLDSVLYDNQE
FPDPEKFKPEHFLNENGKFKYSDYFKPFSTGKRVCAGEGLARMELFLLLCAILQHFNL
KPLVDPKDIDLSPIHIGFGCIPPRYKLCVIPRS
Cytochrome P450, family 2, subfamily C, polypeptide 9
[Homo sapiens]
SEQ ID NO: 7
MDSLVVLVLCLSCLLLLSLWRQSSGRGKLPPGPTPLPVIGNILQIGIKDISKSLTNLSK
VYGPVFTLYFGLKPIVVLHGYEAVKEALIDLGEEFSGRGIFPLAERANRGFGIVFSNG
KKWKEIRRFSLMTLRNFGMGKRSIEDRVQEEARCLVEELRKTKASPCDPTFILGCAP
CNVICSIIFHKRFDYKDQQFLNLMEKLNENIKILSSPWIQICNNFSPIIDYFPGTHNKLL
KNVAFMKSYILEKVKEHQESMDMNNPQDFIDCFLMKMEKEKHNQPSEFTIESLENT
AVDLFGAGTETTSTTLRYALLLLLKHPEVTAKVQEEIERVIGRNRSPCMQDRSHMPY
TDAVVHEVQRYIDLLPTSLPHAVTCDIKFRNYLIPKGTTILISLTSVLHDNKEFPNPEM
FDPHHFLDEGGNFKKSKYFMPFSAGKRICVGEALAGMELFLFLTSILQNFNLKSLVDP
KNLDTTPVVNGFASVPPFYQLCFIPV

All publications and patent documents disclosed or referred to herein are incorporated by reference in their entirety. The foregoing description has been presented only for purposes of illustration and description. This description is not intended to limit the invention to the precise form disclosed. It is intended that the scope of the invention be defined by the claims appended hereto.

What is claimed:

1. A magnetic macroporous powder, comprising a thermoplastic polymer and magnetic microparticles, wherein said powder is operative for additive manufacturing (AM) of a shaped magnetic macroporous scaffold for immobilizing self-assembled mesoporous aggregates of magnetic nanoparticles.

2. The magnetic macroporous powder of claim 1, wherein said magnetic particles have a size of between about 50-100 μm.

3. The magnetic macroporous powder of claim 1, wherein said magnetic particles have a size of between about 10-50 μm.

4. The magnetic macroporous powder of claim 1, wherein said magnetic particles have a size of between about 5-10 μm.

5. The magnetic macroporous powder of claim 1, wherein said magnetic particles have a size of about 10 μm.

6. The magnetic macroporous powder of claim 1, wherein said magnetic particles have a size of about 5 μm.

7. The magnetic macroporous powder of claim 1, wherein said magnetic particles have a size of less than 5 μm.

8. The magnetic macroporous powder of claim 1, wherein said magnetic particles have a size of greater than 100 μm.

9. The magnetic macroporous powder of claim 1 has an average size of about 150 μm.

10. The magnetic macroporous powder of claim 1 has an average size of about 75 μm.

11. The magnetic macroporous powder of claim 1 has an average size of about 15 μm.

12. The magnetic macroporous powder of claim 1, wherein said magnetic particles have a concentration of between 0 and 10% by weight.

13. The magnetic macroporous powder of claim 1, wherein said magnetic particles have a concentration of 10 to 50% by weight.

14. The magnetic macroporous powder of claim 1, wherein said magnetic particles have a concentration of 50 to 90% by weight.

15. The magnetic macroporous powder of claim 1, wherein said thermoplastic polymer comprises a polymer selected from the group consisting of Polyvinyl alcohol (PVA), Acrylic (PMMA), Acrylonitrile butadiene styrene (ABS), Polyamide including Nylon 6 and Nylon 12, Polylactic acid (PLA), Polybenzimidazole (PBI), Polycarbonate (PC), Polyether sulfone (PES), Polyoxymethylene (POM), Polyetherether ketone (PEEK), Polyetherimide (PEI), Polyethylene (PE), Polyphenylene oxide (PEO), Polyphenylene sulfide (PPS), Polypropylene (PP), Polystyrene (PS), Polyvinyl chloride (PVC), polytetrafluoroethylene (PTFE), ω-polyesters, and chemically functionalized derivatives thereof.

16. The magnetic macroporous powder of claim 1, wherein said magnetic microparticles comprise a magnetic material selected from the group consisting of magnetite (Fe₃O₄), hematite (α-Fe₂O₃), maghemite (γ-Fe₂O₃), a spinel ferrite, lodestone, cobalt, nickel, rare earth, and magnetic composites.

17. The magnetic macroporous powder of claim 16, wherein said rare earth is neodymium, gadolinium, sysprosium, samarium-cobalt, or neodymium-iron-boron.

18. The magnetic macroporous powder of claim 16, wherein said magnetic composite comprises a ceramic, ferrite, or alnico magnets.

19. The magnetic macroporous powder of any one of claims 1-18, wherein said thermoplastic polymer and said magnetic microparticles are chemically blended.

20. The magnetic macroporous powder of any one of claims 1-18, wherein said thermoplastic polymer and said magnetic microparticles are thermally blended.

21. The magnetic macroporous powder of any one of claims 1-18, wherein said thermoplastic polymer and said magnetic microparticles are physically blended.

22. The magnetic macroporous powder of any one of claims 1-21, comprising macropores having a size of between 0.5-200 μm.

23. The magnetic macroporous powder of any one of claims 1-22, further comprising cellulose fibers, cellulose nanofibers, glass fibers, or carbon fibers.

24. The magnetic macroporous powder of any one of claims 1-23, further comprising self-assembled mesoporous aggregates of magnetic nanoparticles and an enzyme magnetically immobilized within said mesopores or on their surface.

25. A shaped magnetic macroporous scaffold, comprising the magnetic macroporous powder of any one of claims 1-23, wherein said powder has been formed into said shape by three-dimensional (3D) printing.

26. The shaped magnetic macroporous scaffold of claim 25, wherein said shape is a cylinder, an orb, a bead, a strip, a capsule, a cube, a squared rod, a pyramid, a diamond, a lattice, or an irregular shape.

27. The shaped magnetic macroporous scaffold of either one of claims 25-26, further comprising self-assembled mesoporous aggregates of magnetic nanoparticles.

28. The shaped magnetic macroporous scaffold of claim 27, wherein said self-assembled mesoporous aggregates of magnetic nanoparticles further comprise one or more enzymes magnetically immobilized within said mesopores or on the surface of said magnetic nanoparticles.

29. The shaped magnetic macroporous scaffold of any claim 28, wherein said one or more enzymes are selected from the group consisting of hydrolases, hydroxylases, hydrogen peroxide producing enzymes (HPP), nitralases, hydratases, dehydrogenases, transaminases, ketoreductases (KREDS) ene reductases (EREDS), imine reductases (IREDS), catalases, dismutases, oxidases, dioxygenases, lipoxidases, oxidoreductases, peroxidases, laccases, synthetases, transferases, oxynitrilases, isomerases, gludosidases, kinases, lyases, sucrases, invertases, epimerases, and lipases.

30. The shaped magnetic macroporous scaffold of claim 27, wherein said self-assembled mesoporous aggregates of magnetic nanoparticles comprise microsomes, wherein a first enzyme requiring a diffusible cofactor having a first enzymatic activity is contained within said microsomes, wherein a second enzyme comprising a cofactor regeneration activity is magnetically-entrapped within said mesopores, wherein said cofactor is utilized in said first enzymatic activity; wherein said first and second enzymes function by converting a diffusible substrate into a diffusible product; and wherein said magnetic nanoparticles are magnetically associated with said magnetic macroporous scaffold.

31. The shaped magnetic macroporous scaffold of claim 27, wherein said self-assembled mesoporous aggregates of magnetic nanoparticles comprises a first enzyme requiring a diffusible cofactor having a first enzymatic activity; a second enzyme comprising a cofactor regeneration activity; wherein said cofactor is utilized in said first enzymatic activity; wherein said first and second enzymes are magnetically-entrapped within said mesopores formed by said aggregates of magnetic nanoparticles and said first and second enzymes function by converting a diffusible substrate into a diffusible product.

32. The shaped magnetic macroporous scaffold of claim 31, wherein said first enzyme is an oxidative enzyme.

33. The shaped magnetic macroporous scaffold of claim 32, wherein said oxidative enzyme is a Flavin-containing oxygenase; wherein said composition further comprises a third enzyme having a ω-factor reductase activity that is ω-located with said first enzyme.

34. The shaped magnetic macroporous scaffold of claim 32, wherein said oxidative enzyme is a P450 monooxygenase; wherein said composition further comprises a third enzyme having a ω-factor reductase activity that is ω-located with said first enzyme.

35. The shaped magnetic macroporous scaffold of claim 32, wherein said P450 monooxygenase and said third enzyme are comprised within a single protein.

36. The shaped magnetic macroporous scaffold of claim 35, wherein said single protein comprises a bifunctional cytochrome P450/NADPH—P450 reductase.

37. The shaped magnetic macroporous scaffold of claim 35, wherein said single protein has BM3 activity and has at least a 90% sequence identity to SEQ ID NO:1.

38. The shaped magnetic macroporous scaffold of any one of claims 25-37, wherein said scaffold is formed in a shape suited for a particular biocatalytic process.

39. A method of making a shaped magnetic macroporous scaffold comprising the magnetic macroporous powder of any one of claims 1-23, comprising additively manufacturing (AM) said shaped magnetic macroporous scaffold using a three-dimensional (3D) printer, wherein said shape is taken from a 3D model.

40. The method of claim 39, wherein said 3D model is an electronic file.

41. The method of claim 40, wherein said electronic file is a computer-aided design (CAD) or a stereolithography (STL) file.

42. The method of any one of claims 39-41, wherein said AM is Fused Filament Fabrication (FFF) or Selective laser sintering (SLS).

43. The method of any one of claims 39-42, wherein said macropores are formed using a soluble agent selected from the group consisting of a salt, a sugar, or a small soluble polymer, and removing said soluble agent with a solvent.

44. A method of making a device for catalyzing an enzymatic reaction, comprising combining a shaped magnetic macroporous scaffold with self-assembled mesoporous aggregates of magnetic nanoparticles and an enzyme, wherein said enzyme is magnetically immobilized within said mesopores.

45. A method of catalyzing a reaction between a plurality of substrates, comprising exposing said magnetic macroporous powder of claim 24 to said substrates under conditions in which said enzyme catalyzes said reaction between said substrates.

46. A method of catalyzing a reaction between a plurality of substrates, comprising exposing said shaped magnetic macroporous scaffold of either one of claims 28-29 to said substrates under conditions in which said enzyme catalyzes said reaction between said substrates.

47. The method of either one of claims 45-46, wherein said reaction is used in the manufacture of a pharmaceutical product.

48. The method of either one of claims 45-46, wherein said reaction is used in the manufacture of a medicament.

49. The method of either one of claims 45-46, wherein said reaction is used in the manufacture of a food product.

50. The method of either one of claims 45-46, wherein said reaction is used in the manufacture of a flavor.

51. The method of either one of claims 45-46, wherein said reaction is used in the manufacture of a fragrance.

52. The method of either one of claims 45-46, wherein said reaction is used in the manufacture of a sweetener.

53. The method of either one of claims 45-46, wherein said reaction is used in the manufacture of an agrochemical.

54. The method of either one of claims 45-46, wherein said reaction is used in the manufacture of an antimicrobial agent.

55. The method of either one of claims 45-46, wherein said reaction is used in the manufacture of a toxin.

56. The method of either one of claims 45-46, wherein said reaction is used in the manufacture of a detergent.

57. The method of either one of claims 45-46, wherein said reaction is used in the manufacture of a fuel product.

58. The method of either one of claims 45-46, wherein said reaction is used in the manufacture of a biochemical product.

59. The method of either one of claims 45-46, wherein said reaction is used in the manufacture of a paper product.

60. The method of either one of claims 45-46, wherein said reaction is used in the manufacture of a plastic product.

61. The method of either one of claims 45-46, wherein said reaction is used in a process for removing a contaminant from a solution.

62. The method of claim 61, wherein said solution is an aqueous solution, a solvent, or an oil.

63. The method of either one of claims 45-46, further comprising the step of removing said mesoporous aggregates and replacing them with a fresh preparation of mesoporous aggregates.

64. The method of any one of claims 45-46, wherein said method is carried out using flow cell and continuous manufacturing.
